(19) United States
(12) Patent Application Publication
(10) Pub. No.: US 2023/0127898 A1
(43) Pub. Date: Apr. 27, 2023

(54) HEPATITIS B CAPSID ASSEMBLY MODULATORS

(71) Applicant: Venatorx Pharmaceuticals, Inc., Malvem, PA (US)

(72) Inventors: Jiangchao YAO, Princeton, NJ (US); Glen COBURN, Bethel, CT (US); Bin LIU, Plainsboro, NJ (US); Christopher BENETATOS, Chester Springs, PA (US); Steven A. BOYD, Chester Springs, PA (US); Stephen M. CONDON, Glenmoore, PA (US); Thomas HAIMOWITZ, Newton, PA (US)

(21) Appl. No.: 17/908,752

(22) PCT Filed: Mar. 3, 2021
(86) PCT No.: PCT/US2021/020397

§ 371 Date: Sep. 1, 2022

Related U.S. Application Data
(60) Provisional application No. 62/984,682, filed on Mar. 3, 2020.

(51) Int. Cl. C07D 487/04; C07D 498/14; C07D 498/04; A61P 31/20; A61K 45/06; C07D 471/04

(52) U.S. Cl. CPC C07D 487/04; C07D 498/14; C07D 498/04; A61P 31/20; A61K 45/06; C07D 471/04

(57) ABSTRACT

Described herein are hepatitis B capsid assembly modulator compounds of formula (I) and pharmaceutical compositions comprising said compounds. The subject compounds and compositions are useful for the treatment of hepatitis B.

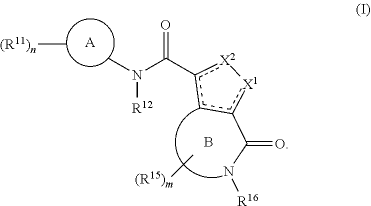

### CROSS-REFERENCE

This application claims the benefit of U.S. Provisional Application Ser. No. 62/984,682 filed Mar. 3, 2020 which is hereby incorporated by reference in its entirety.

### BACKGROUND OF THE INVENTION

HBV is a small enveloped DNA virus belonging to the Hepadnaviridae family that is distributed worldwide as ten geographically distinct genotypes. Infection with HBV is typically self-limiting in otherwise healthy adults; however, vertical transmission or exposure during early childhood often results in a chronic lifelong infection. Worldwide there are an estimated >400 million individuals chronically infected with HBV that are at risk for complications due to liver disease, including cirrhosis, fibrosis, hepatocellular carcinoma and death. Each year 500,000 to 1 million people die from end stage liver disease as a consequence of HBV infection.

The compact HBV genome utilizes four overlapping reading frames to encode the major structural and non-structural proteins: polymerase (F), envelope (S), core (C) and the X protein (X). HBV enters human hepatocytes via receptor mediated endocytosis, following binding of the envelope glycoprotein to its primary receptor, the bile acid transporter sodium taurocholate co-transporting polypeptide (NTCP). Following fusion with the endosome membrane, the capsid is ejected into the cytoplasm and translocated to the nucleus. The partially double-stranded, relaxed, circular HBV genome (RC DNA) is converted to a covalently closed circular DNA form (cccDNA) by host cellular DNA repair mechanisms. The HBV cccDNA serves as the template for RNA polymerase II-dependent transcription of multiple RNA species, including viral mRNAs and the 3.2-kbp pre-genomic RNA (pgRNA). During the maturation process, pgRNA is packaged into capsids along with the HBV polymerase. The pgRNA is then reverse transcribed into a negative-stranded DNA template that is subsequently converted into the partially double-stranded RC DNA species by the polymerase. Mature, enveloped HBV particles containing the RC DNA genome are secreted from the surface of the infected hepatocyte ready to initiate new cycles of infection.

The capsid is composed of 240 copies of the core protein that spontaneously self-assemble through a network of weak inter-subunit interactions. In vitro evidence suggests that a trimer of core dimers initiates the nucleation event that rapidly recruits additional dimers to form the icosahedral core structure (T=4). In addition to its structural role, encapsidation of the pgRNA is an essential step required for HBV DNA synthesis and formation of the mature capsid particle. The core protein also plays an important role in shuttling the RC DNA into the nucleus to initiate and maintain the cccDNA pools and may also play a role in regulating interferon sensitive gene expression. Thus, capsid modulators may have the unique ability to intervene at multiple points in the HBV lifecycle.

Several chemotype series of HBV capsid assembly modulators have been reported in the literature including: phenylpropenamides (PP) (e.g., AT-130), heteroarylpyrimidines (HAP) (e.g. Bay 41-4109), and sulfamoylbenzamides (SBA) (e.g. NVR 3-778). Capsid modulators exert their effects on the assembly process through one of two different mechanisms of action. The HAP series induces the aberrant assembly of large capsid aggregates that subsequently triggers the degradation of the core protein. The PP and SBA series, on the other hand, appear to accelerate capsid assembly resulting in the production of authentic empty capsid particles that have failed to incorporate pgRNA. Assembly modulators representing both mechanisms have demonstrated the ability to reduce HBV DNA levels in mouse models of infection. More recently, NVR 3-778 (SBA) demonstrated clinical proof-of-concept in a Phase 1b clinical trial, resulting in a −1.7 log 10 reduction in HBV DNA following 600 mg bid dosing for 29 days. The need remains for new antiviral HBV capsid modulators with improved properties for the treatment of chronic hepatitis B.

### SUMMARY OF THE INVENTION

The present disclosure relates to small-molecule compounds that modulate capsid assembly and block hepatitis B virus (HBV) replication with the potential to be used as a monotherapy or in combination with additional antivirals and/or other anti-HBV agents that are useful for the treatment of chronic HBV infection.

Described herein are compounds of Formula (I), (Ia), or (Ib) that modulate the normal capsid assembly of hepatitis B core proteins to inhibit the hepatitis B lifecycle, and thus act as antiviral agents toward HBV.

Disclosed herein are compounds of Formula (I), or a pharmaceutically acceptable salt, solvate, or stereoisomer thereof:

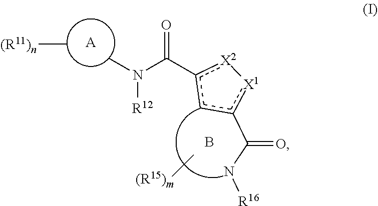

wherein:
Ring A is aryl, heteroaryl, cycloalkyl, or heterocycloalkyl;
each R¹¹ is independently halogen, —CN, —OH, —OR^{a}, —SH, —SR^{a}, —S(═O)R^{a}, —S(═O)₂R^{a}, —NO₂, —NR^{c}R^{d}, —NR^{b}S(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is independently optionally substituted with one, two, or three R¹;
or two R¹¹ on adjacent atoms are taken together with the atoms to which they are attached to form a cycloalkyl, heterocycloalkyl, aryl, or heteroaryl; each optionally substituted with one, two, or three R²;
n is 0-4;
R¹² is hydrogen or C₁-C₆alkyl;

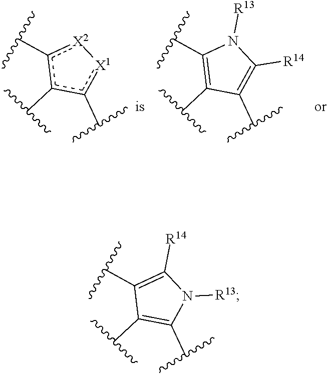

R¹³ is hydrogen, —S(═O)R^{a}, —S(═O)₂R^{a}, —S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein the alkyl, alkenyl, alkynyl, cycloalkyl, heterocycloalkyl, aryl, and heteroaryl is optionally substituted with one, two, or three R³;
R¹⁴ is hydrogen, halogen, —CN, —OH, —OR^{a}, —SH, —SR^{a}, —S(═O)R^{a}, —S(═O)₂R^{a}, —NO₂, —NR^{c}R^{d}, —NRS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein the alkyl, alkenyl, alkynyl, cycloalkyl, heterocycloalkyl, aryl, and heteroaryl is optionally substituted with one, two, or three R⁴;
Ring B is a 6- to 9-membered heterocycloalkyl;
each R¹⁵ is independently halogen, —CN, —OH, —OR^{a}, —SH, —SR^{a}, —S(═O)R^{a}, —S(═O)₂R^{a}, —NO₂, —NR^{c}R^{d}, —NRS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is independently optionally substituted with one, two, or three R⁵; or
two R¹⁵ on the same carbon atom are taken together to form an oxo, a C₂-C₆alkene, a cycloalkyl, or a heterocycloalkyl; wherein the cycloalkyl and heterocycloalkyl is optionally substituted with one, two, or three R⁶; or
two R¹⁵ on adjacent atoms are taken together to form a cycloalkyl or a heterocycloalkyl; wherein the cycloalkyl and heterocycloalkyl is optionally substituted with one, two, or three R⁷; or
two R¹⁵ on adjacent atoms are taken together to form a double bond;
m is 0-6;
R¹⁶ is hydrogen or C₁-C₆alkyl;
each R¹, R², R³, R⁴, R⁵, R⁶, and R⁷ is independently halogen, —CN, —OH, —OR^{a}, —SH, —SR^{a}, —S(═O)R^{a}, —S(═O)₂R^{a}, —NO₂, —NR^{c}R^{d}, —NR^{b}S(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl;
or two R¹, two R², two R³, two R⁴, two R⁵, two R⁶, or two R⁷ on the same carbon are taken together to form an oxo;
each R^{a} is independently C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl;
each R^{b} is independently hydrogen, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl;
or R^{c} and R^{d} are taken together with the atom to which they are attached to form a heterocycloalkyl optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl.

Also disclosed herein is a pharmaceutical composition comprising a compound disclosed herein, or a pharmaceutically acceptable salt, solvate, or stereoisomer thereof, and a pharmaceutically acceptable excipient.

Also disclosed herein is a method of treating an infection in a subject, comprising administering to the subject a compound disclosed herein, or a pharmaceutically acceptable salt, solvate, or stereoisomer thereof.

Also disclosed herein is a method of treating an infection in a subject, comprising administering to the subject a pharmaceutical composition disclosed herein.

In some embodiments, the infection is a viral infection.

In some embodiments, the infection is caused by the hepatitis B virus.

In some embodiments, the infection is hepatitis B.

In some embodiments, the method further comprises administering an additional therapeutic agent useful for treating a chronic HBV infection.

In some embodiments, the additional therapeutic agent useful for treating a chronic HBV infection is a reverse transcriptase inhibitor; an HBV polymerase inhibitor, a capsid inhibitor; a cccDNA formation inhibitor; an RNA destabilizer; a checkpoint inhibitor (PD-1/PD-L1 inhibitor); a therapeutic vaccine; an RNA interference (RNAi) therapeutic; an antisense-based therapeutic, an HBV entry inhibitor; a TLR agonist; an RIG-I agonist, or an interferon.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Chronic hepatitis B infection (CHB) is currently managed with interferon-alpha or nucleoside(tide) analog-based therapies that target the HBV encoded polymerase/reverse transcriptase. The effectiveness of interferon-alpha is limited by inadequate long term responses and severe side effects, while entecavir and tenofovir, are generally well-tolerated, possess a high barrier to resistance and potently suppress viral replication. None of the aforementioned frontline therapies are curative, however, and expensive lifelong therapy is required to maintain a virologic response and prevent the complications associated with liver disease. Novel therapies representing different treatment classes are therefore urgently required to improve functional cure rates (i.e. defined as the loss of HBsAg expression) and shorten treatment durations. Modulators of HBV capsid assembly represent one such class of antivirals with the potential to improve outcomes for chronically infected individuals.

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkenyl is optionally substituted with oxo, halogen, —CN, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkynyl is optionally substituted with oxo, halogen, —CN, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkylene is optionally substituted with oxo, halogen, —CN, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkoxy is optionally substituted with halogen, —CN, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, the aryl is optionally substituted with halogen, methyl, ethyl, —CN, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic or polycyclic carbocyclic ring, which may include fused (when fused with an aryl or a heteroaryl ring, the cycloalkyl is bonded through a non-aromatic ring atom) or bridged ring systems. In some embodiments, the cycloalkyl is fully saturated. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 10-membered cycloalkyl. In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl), from two to ten carbon atoms (C₂-C₁₀ heterocycloalkyl), from two to eight carbon atoms (C₂-C₈ heterocycloalkyl), from two to seven carbon atoms (C₂-C₇ heterocycloalkyl), from two to six carbon atoms (C₂-C₆ heterocycloalkyl), from two to five carbon atoms (C₂-C₅ heterocycloalkyl), or two to four carbon atoms (C₂-C₄ heterocycloalkyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, the heterocycloalkyl is optionally substituted with oxo, halogen, methyl, ethyl, —CN, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, the heteroaryl is optionally substituted with halogen, methyl, ethyl, —CN, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition. In some embodiments, treatment also includes prophylactic treatment (e.g., administration of a composition described herein when an individual is suspected to be suffering from a viral infection, e.g., hepatitis B).

“Capsid inhibitor” includes compounds that are capable of inhibiting the expression and/or function of a capsid protein either directly or indirectly.

“HBV infection” refers to any and all conditions deriving from infection with HBV, including but not limited to hepatitis B, preferably chronic hepatitis B, HBV/HDV co-infection, HBV/HCV coinfection, HBV/HIV coinfection.

As used herein “reducing reoccurrence of an HBV infection” indicates that patients may have reactivation of HBV replication and exacerbation of a condition related to an HBV infection, e.g. hepatitis, after years of quiescence. These patients may still be at risk of developing a condition related to an HBV infection, e.g. hepatocellular carcinoma development. Antiviral therapy is also recommended as prophylaxis for patients who are HBsAg-positive as well as patients who are HBsAg-negative and hepatitis B core antibody-positive who require treatment with immunosuppressive therapies that are predicted to have a moderate to high risk of HBV reactivation.

### Compounds

Described herein are compounds of Formula (I), (Ia), or (Ib), or a pharmaceutically acceptable salt, solvate, or stereoisomer thereof useful in the treatment of viral infections. In some embodiments, the viral infection is a chronic hepatitis B infection.

Disclosed herein is a compound of Formula (I), or a pharmaceutically acceptable salt, solvate, or stereoisomer thereof:

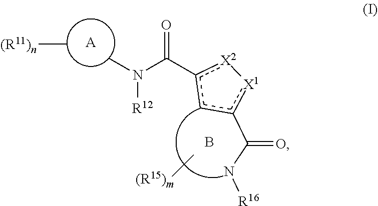

wherein:
Ring A is aryl, heteroaryl, cycloalkyl, or heterocycloalkyl;
each R¹¹ is independently halogen, —CN, —OH, —OR^{a}, —SH, —SR^{a}, —S(═O)R^{a}, —S(═O)₂R^{a}, —NO₂, —NR^{c}R^{d}, —NR^{b}S(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is independently optionally substituted with one, two, or three R¹;
or two R¹¹ on adjacent atoms are taken together with the atoms to which they are attached to form a cycloalkyl, heterocycloalkyl, aryl, or heteroaryl; each optionally substituted with one, two, or three R²;
n is 0-4;
R¹² is hydrogen or C₁-C₆alkyl;

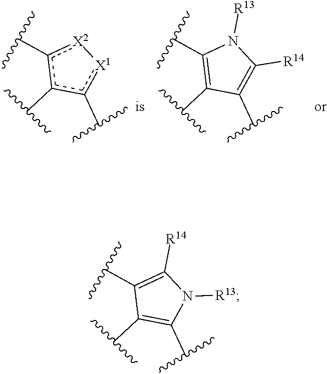

R¹³ is hydrogen, —S(═O)R^{a}, —S(═O)₂R^{a}, —S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein the alkyl, alkenyl, alkynyl, cycloalkyl, heterocycloalkyl, aryl, and heteroaryl is optionally substituted with one, two, or three R³;
R¹⁴ is hydrogen, halogen, —CN, —OH, —OR^{a}, —SH, —SR^{a}, —S(═O)R^{a}, —S(═O)₂R^{a}, —NO₂, —NR^{c}R^{d}, —NR^{b}S(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein the alkyl, alkenyl, alkynyl, cycloalkyl, heterocycloalkyl, aryl, and heteroaryl is optionally substituted with one, two, or three R⁴;
Ring B is a 6- to 9-membered heterocycloalkyl;
each R¹⁵ is independently halogen, —CN, —OH, —OR^{a}, —SH, —SR^{a}, —S(═O)R^{a}, —S(═O)₂R^{a}, —NO₂, —NR^{c}R^{d}, —NRS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is independently optionally substituted with one, two, or three R⁵; or
two R¹⁵ on the same carbon atom are taken together to form an oxo, a C₂-C₆alkene, a cycloalkyl, or a heterocycloalkyl; wherein the cycloalkyl and heterocycloalkyl is optionally substituted with one, two, or three R⁶; or
two R¹⁵ on adjacent atoms are taken together to form a cycloalkyl or a heterocycloalkyl; wherein the cycloalkyl and heterocycloalkyl is optionally substituted with one, two, or three R⁷; or
two R¹⁵ on adjacent atoms are taken together to form a double bond;
m is 0-6;
R¹⁶ is hydrogen or C₁-C₆alkyl;
each R¹, R², R³, R⁴, R⁵, R⁶, and R⁷ is independently halogen, —CN, —OH, —OR^{a}, —SH, —SR^{a}, —S(═O)R^{a}, —S(═O)₂R^{a}, —NO₂, —NR^{c}R^{d}, —NR^{b}S(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl;
or two R¹, two R², two R³, two R⁴, two R⁵, two R⁶, or two R⁷ on the same carbon are taken together to form an oxo;
each R^{a} is independently C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl;
each R^{b} is independently hydrogen, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl;
or R^{c} and R^{d} are taken together with the atom to which they are attached to form a heterocycloalkyl optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl.

In some embodiments of a compound of Formula (I), is

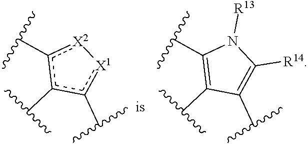

In some embodiments of a compound of Formula (I), is

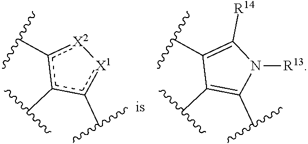

In some embodiments the compound of Formula (I), or a pharmaceutically acceptable salt, solvate, or stereoisomer thereof, is of Formula (Ia):

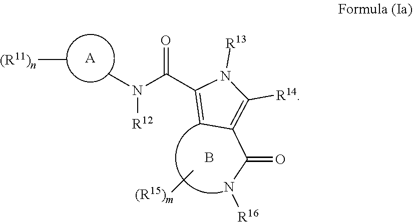

In some embodiments the compound of Formula (I), or a pharmaceutically acceptable salt, solvate, or stereoisomer thereof, is of Formula (Ib):

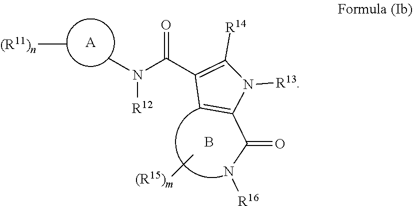

In some embodiment of a compound of Formula (I), (Ia), or (Ib), Ring A is cycloalkyl or heterocycloalkyl. In some embodiment of a compound of Formula (I), (Ia), or (Ib), Ring A is cycloalkyl, aryl or heteroaryl. In some embodiment of a compound of Formula (I), (Ia), or (Ib), Ring A is aryl or heteroaryl. In some embodiment of a compound of Formula (I), (Ia), or (Ib), Ring A is phenyl or 5- or 6-membered heteroaryl. In some embodiment of a compound of Formula (I), (Ia), or (Ib), Ring A is phenyl or 6-membered heteroaryl. In some embodiment of a compound of Formula (I), (Ia), or (Ib), Ring A is phenyl or pyridyl. In some embodiment of a compound of Formula (I), (Ia), or (Ib), Ring A is phenyl. In some embodiment of a compound of Formula (I), (Ia), or (Ib), Ring A is pyridyl.

In some embodiment of a compound of Formula (I), (Ia), or (Ib), n is 0-3. In some embodiment of a compound of Formula (I), (Ia), or (Ib), n is 0-2. In some embodiment of a compound of Formula (I), (Ia), or (Ib), n is 0 or 1. In some embodiment of a compound of Formula (I), (Ia), or (Ib), n is 1-3. In some embodiment of a compound of Formula (I), (Ia), or (Ib), n is 1 or 2. In some embodiment of a compound of Formula (I), (Ia), or (Ib), n is 0. In some embodiment of a compound of Formula (I), (Ia), or (Ib), n is 1. In some embodiment of a compound of Formula (I), (Ia), or (Ib), n is 2. In some embodiment of a compound of Formula (I), (Ia), or (Ib), n is 3. In some embodiment of a compound of Formula (I), (Ia), or (Ib), n is 4.

In some embodiment of a compound of Formula (I), (Ia), or (Ib), each R¹¹ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is independently optionally substituted with one, two, or three R¹. In some embodiment of a compound of Formula (I), (Ia), or (Ib), each R¹¹ is independently halogen, —CN, C₁-C₆alkyl, C₁-C₆haloalkyl, C₁-C₆hydroxyalkyl, C₁-C₆aminoalkyl, or cycloalkyl; wherein each alkyl and cycloalkyl is independently optionally substituted with one, two, or three R¹. In some embodiment of a compound of Formula (I), (Ia), or (Ib), each R¹¹ is independently halogen, —CN, C₁-C₆alkyl, C₁-C₆haloalkyl, C₁-C₆hydroxyalkyl, C₁-C₆aminoalkyl; wherein each alkyl is independently optionally substituted with one, two, or three R¹. In some embodiment of a compound of Formula (I), (Ia), or (Ib), each R¹¹ is independently halogen, —CN, C₁-C₆alkyl, or C₁-C₆haloalkyl; wherein each alkyl is independently optionally substituted with one, two, or three R¹. In some embodiment of a compound of Formula (I), (Ia), or (Ib), each R¹¹ is independently halogen, —CN, —OH, —OR^{a}, —NR^{c}R^{d}, —C(═O)OR^{b}, —C(═O)NR^{c}R^{d}, C₁-C₆alkyl, C₁-C₆haloalkyl, or cycloalkyl. In some embodiment of a compound of Formula (I), (Ia), or (Ib), each R¹¹ is independently halogen or C₁-C₆alkyl. In some embodiments of a compound of Formula (I), (Ia), or (Ib), each R¹¹ is independently halogen, —CN, —OH, —OR^{a}, —NR^{c}R^{d}, —C(═O)OR^{b}, —C(═O)NR^{c}R^{d}, C₁-C₆alkyl, C₁-C₆haloalkyl, or cycloalkyl.

In some embodiment of a compound of Formula (I), (Ia), or (Ib), each alkyl, alkenyl, alkynyl, cycloalkyl, heterocycloalkyl, aryl, and heteroaryl in R¹¹ is independently optionally substituted with one, two, or three R¹. In some embodiment of a compound of Formula (I), (Ia), or (Ib), each alkyl, alkenyl, alkynyl, cycloalkyl, heterocycloalkyl, aryl, and heteroaryl in R¹¹ is independently optionally substituted with one or two R¹. In some embodiment of a compound of Formula (I), (Ia), or (Ib), each alkyl, alkenyl, alkynyl, cycloalkyl, heterocycloalkyl, aryl, and heteroaryl in R¹¹ is independently optionally substituted with one R¹. In some embodiment of a compound of Formula (I), (Ia), or (Ib), each alkyl, alkenyl, alkynyl, cycloalkyl, heterocycloalkyl, aryl, and heteroaryl in R¹¹ is independently optionally substituted with two R¹. In some embodiment of a compound of Formula (I), (Ia), or (Ib), each alkyl, alkenyl, alkynyl, cycloalkyl, heterocycloalkyl, aryl, and heteroaryl in R¹¹ is independently optionally substituted with three R¹.

In some embodiment of a compound of Formula (I), (Ia), or (Ib), each R¹ is independently halogen, —CN, —OH, —OR^{a}, —NR^{c}R^{d}, —C(═O)R^{a}, —C(═O)OR^{b}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is independently optionally substituted with one, two, or three oxo, halogen, —CN, —OH, —OMe, —NH₂, C₁-C₆alkyl, C₁-C₆haloalkyl, C₁-C₆hydroxyalkyl, or C₁-C₆aminoalkyl. In some embodiment of a compound of Formula (I), (Ia), or (Ib), each R¹ is independently oxo, halogen, —CN, —OH, —OR^{a}, —NR^{c}R^{d}, C₁-C₆alkyl, C₁-C₆haloalkyl, C₁-C₆hydroxyalkyl, or C₁-C₆aminoalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), (Ia), or (Ib), each R¹ is independently oxo, halogen, —CN, —OH, —OR^{a}, —NR^{c}R^{d}, C₁-C₆alkyl, C₁-C₆haloalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), (Ia), or (Ib), each R¹ is independently oxo, halogen, —CN, —OH, —OR^{a}, —NR^{c}R^{d}, C₁-C₆alkyl, C₁-C₆haloalkyl. In some embodiment of a compound of Formula (I), (Ia), or (Ib), each R¹ is independently oxo, halogen, —CN, —OH, —OMe, —NH₂, Me, or CF₃. In some embodiment of a compound of Formula (I), (Ia), or (Ib), each R¹ is independently halogen.

In some embodiment of a compound of Formula (I), (Ia), or (Ib), two R¹¹ on adjacent atoms are taken together with the atoms to which they are attached to form a cycloalkyl, heterocycloalkyl, aryl, or heteroaryl; each optionally substituted with one, two, or three R². In some embodiment of a compound of Formula (I), (Ia), or (Ib), two R¹¹ on adjacent atoms are taken together with the atoms to which they are attached to form a cycloalkyl optionally substituted with one, two, or three R². In some embodiment of a compound of Formula (I), (Ia), or (Ib), two R¹¹ on adjacent atoms are taken together with the atoms to which they are attached to form a heterocycloalkyl optionally substituted with one, two, or three R². In some embodiment of a compound of Formula (I), (Ia), or (Ib), two R¹¹ on adjacent atoms are taken together with the atoms to which they are attached to form an aryl optionally substituted with one, two, or three R². In some embodiment of a compound of Formula (I), (Ia), or (Ib), two R¹¹ on adjacent atoms are taken together with the atoms to which they are attached to form a heteroaryl optionally substituted with one, two, or three R².

In some embodiment of a compound of Formula (I), (Ia), or (Ib), the cycloalkyl, heterocycloalkyl, aryl, and heteroaryl formed when two R¹¹ are taken together is optionally substituted with one, two, or three R². In some embodiment of a compound of Formula (I), (Ia), or (Ib), the cycloalkyl, heterocycloalkyl, aryl, and heteroaryl formed when two R¹¹ are taken together is optionally substituted with one or two R². In some embodiment of a compound of Formula (I), (Ia), or (Ib), the cycloalkyl, heterocycloalkyl, aryl, and heteroaryl formed when two R¹¹ are taken together is optionally substituted with one R². In some embodiment of a compound of Formula (I), (Ia), or (Ib), the cycloalkyl, heterocycloalkyl, aryl, and heteroaryl formed when two R¹¹ are taken together is optionally substituted with two R². In some embodiment of a compound of Formula (I), (Ia), or (Ib), the cycloalkyl, heterocycloalkyl, aryl, and heteroaryl formed when two R¹¹ are taken together is optionally substituted with three R².

In some embodiment of a compound of Formula (I), (Ia), or (Ib), each R² is independently halogen, —CN, —OH, —OR^{a}, —NR^{c}R^{d}, —C(═O)R^{a}, —C(═O)OR^{b}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is independently optionally substituted with one, two, or three oxo, halogen, —CN, —OH, —OMe, —NH₂, C₁-C₆alkyl, C₁-C₆haloalkyl, C₁-C₆hydroxyalkyl, or C₁-C₆aminoalkyl. In some embodiment of a compound of Formula (I), (Ia), or (Ib), each R² is independently oxo, halogen, —CN, —OH, —OR^{a}, —NR^{c}R^{d}, C₁-C₆alkyl, C₁-C₆haloalkyl, C₁-C₆hydroxyalkyl, or C₁-C₆aminoalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), (Ia), or (Ib), each R² is independently oxo, halogen, —CN, —OH, —OR^{a}, —NR^{c}R^{d}, C₁-C₆alkyl, C₁-C₆haloalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), (Ia), or (Ib), each R² is independently oxo, halogen, —CN, —OH, —OR^{a}, —NR^{c}R^{d}, C₁-C₆alkyl, C₁-C₆haloalkyl. In some embodiment of a compound of Formula (I), (Ia), or (Ib), each R² is independently oxo, halogen, —CN, —OH, —OMe, —NH₂, Me, or CF₃. In some embodiment of a compound of Formula (I), (Ia), or (Ib), each R² is independently halogen.

In some embodiment of a compound of Formula (I), (Ia), or (Ib),

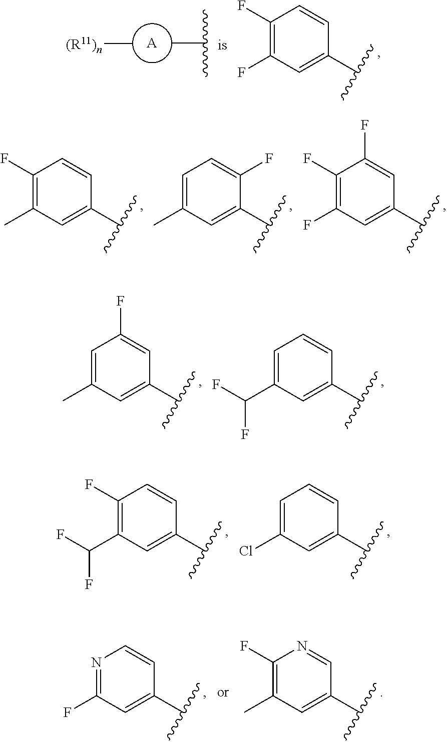

In some embodiment of a compound of Formula (I), (Ia), or (Ib),

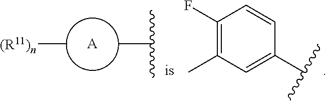

In some embodiment of a compound of Formula (I), (Ia), or (Ib), R¹² is hydrogen. In some embodiment of a compound of Formula (I), (Ia), or (Ib), R¹² is C₁-C₆alkyl.

In some embodiment of a compound of Formula (I), (Ia), or (Ib), R¹³ is hydrogen, —S(═O)₂R^{a}, —S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, cycloalkyl, or heterocycloalkyl; wherein the alkyl, cycloalkyl, and heterocycloalkyl is optionally substituted with one, two, or three R³. In some embodiment of a compound of Formula (I), (Ia), or (Ib), R¹³ is hydrogen, —S(═O)₂R^{a}, —S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, or C₁-C₆aminoalkyl; wherein the alkyl is optionally substituted with one, two, or three R³. In some embodiment of a compound of Formula (I), (Ia), or (Ib), R¹³ is hydrogen, C₁-C₆alkyl, C₁-C₆haloalkyl, C₁-C₆hydroxyalkyl, or C₁-C₆aminoalkyl; wherein the alkyl is optionally substituted with one, two, or three R³. In some embodiment of a compound of Formula (I), (Ia), or (Ib), R¹³ is hydrogen, —S(═O)₂R^{a}, —S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, or cycloalkyl. In some embodiment of a compound of Formula (I), (Ia), or (Ib), R¹³ is hydrogen, C₁-C₆alkyl, C₁-C₆haloyalkyl, or C₁-C₆hydroxyalkyl. In some embodiment of a compound of Formula (I), (Ia), or (Ib), R¹³ is hydrogen, C₁-C₆alkyl, or C₁-C₆hydroxyalkyl. In some embodiment of a compound of Formula (I), (Ia), or (Ib), R¹³ is hydrogen or C₁-C₆alkyl. In some embodiment of a compound of Formula (I), (Ia), or (Ib), R¹³ is C₁-C₆alkyl. In some embodiment of a compound of Formula (I), (Ia), or (Ib), R¹³ is hydrogen.

In some embodiment of a compound of Formula (I), (Ia), or (Ib), the alkyl, alkenyl, alkynyl, cycloalkyl, heterocycloalkyl, aryl, and heteroaryl in R¹³ is optionally substituted with one, two, or three R³. In some embodiment of a compound of Formula (I), (Ia), or (Ib), the alkyl, alkenyl, alkynyl, cycloalkyl, heterocycloalkyl, aryl, and heteroaryl in R¹³ is optionally substituted with one or two R³. In some embodiment of a compound of Formula (I), (Ia), or (Ib), the alkyl, alkenyl, alkynyl, cycloalkyl, heterocycloalkyl, aryl, and heteroaryl is R¹³ in optionally substituted with one R³. In some embodiment of a compound of Formula (I), (Ia), or (Ib), the alkyl, alkenyl, alkynyl, cycloalkyl, heterocycloalkyl, aryl, and heteroaryl in R¹³ is optionally substituted with two R³. In some embodiment of a compound of Formula (I), (Ia), or (Ib), the alkyl, alkenyl, alkynyl, cycloalkyl, heterocycloalkyl, aryl, and heteroaryl in R¹³ is optionally substituted with three R³.

In some embodiment of a compound of Formula (I), (Ia), or (Ib), each R³ is independently halogen, —CN, —OH, —OR^{a}, —NR^{c}R^{d}, —C(═O)R^{a}, —C(═O)OR^{b}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is independently optionally substituted with one, two, or three oxo, halogen, —CN, —OH, —OMe, —NH₂, C₁-C₆alkyl, C₁-C₆haloalkyl, C₁-C₆hydroxyalkyl, or C₁-C₆aminoalkyl. In some embodiment of a compound of Formula (I), (Ia), or (Ib), each R³ is independently oxo, halogen, —CN, —OH, —OR^{a}, —NR^{c}R^{d}, C₁-C₆alkyl, C₁-C₆haloalkyl, C₁-C₆hydroxyalkyl, or C₁-C₆aminoalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), (Ia), or (Ib), each R³ is independently oxo, halogen, —CN, —OH, —OR^{a}, —NR^{c}R^{d}, C₁-C₆alkyl, C₁-C₆haloalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), (Ia), or (Ib), each R³ is independently oxo, halogen, —CN, —OH, —OR^{a}, —NR^{c}R^{d}, C₁-C₆alkyl, C₁-C₆haloalkyl. In some embodiment of a compound of Formula (I), (Ia), or (Ib), each R³ is independently oxo, halogen, —CN, —OH, —OMe, —NH₂, Me, or CF₃. In some embodiment of a compound of Formula (I), (Ia), or (Ib), each R³ is independently halogen.

In some embodiment of a compound of Formula (I), (Ia), or (Ib), R¹⁴ is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, cycloalkyl, or heterocycloalkyl; wherein the alkyl, cycloalkyl, and heterocycloalkyl is optionally substituted with one, two, or three R⁴. In some embodiment of a compound of Formula (I), (Ia), or (Ib), R¹⁴ is hydrogen, halogen, —CN, —OH, —OR^{a}, —NR^{c}R^{d}, C₁-C₆alkyl, C₁-C₆haloalkyl, C₁-C₆hydroxyalkyl, or C₁-C₆aminoalkyl; wherein the alkyl is optionally substituted with one, two, or three R⁴. In some embodiment of a compound of Formula (I), (Ia), or (Ib), R¹⁴ is hydrogen, halogen, C₁-C₆alkyl, C₁-C₆haloalkyl, C₁-C₆hydroxyalkyl, or C₁-C₆aminoalkyl; wherein the alkyl is optionally substituted with one, two, or three R⁴. In some embodiment of a compound of Formula (I), (Ia), or (Ib), R¹⁴ is hydrogen, halogen, C₁-C₆alkyl, C₁-C₆haloalkyl; wherein the alkyl is optionally substituted with one, two, or three R⁴. In some embodiment of a compound of Formula (I), (Ia), or (Ib), R¹⁴ is hydrogen, halogen, or C₁-C₆alkyl optionally substituted with one, two, or three R⁴. In some embodiment of a compound of Formula (I), (Ia), or (Ib), R¹⁴ is hydrogen or C₁-C₆alkyl. In some embodiment of a compound of Formula (I), (Ia), or (Ib), R¹⁴ is C₁-C₆alkyl.

In some embodiment of a compound of Formula (I), (Ia), or (Ib), the alkyl, alkenyl, alkynyl, cycloalkyl, heterocycloalkyl, aryl, and heteroaryl in R¹⁴ is optionally substituted with one, two, or three R⁴. In some embodiment of a compound of Formula (I), (Ia), or (Ib), the alkyl, alkenyl, alkynyl, cycloalkyl, heterocycloalkyl, aryl, and heteroaryl in R¹⁴ is optionally substituted with one or two R⁴. In some embodiment of a compound of Formula (I), (Ia), or (Ib), the alkyl, alkenyl, alkynyl, cycloalkyl, heterocycloalkyl, aryl, and heteroaryl in R¹⁴ is optionally substituted with one R⁴. In some embodiment of a compound of Formula (I), (Ia), or (Ib), the alkyl, alkenyl, alkynyl, cycloalkyl, heterocycloalkyl, aryl, and heteroaryl in R¹⁴ is optionally substituted with two R⁴. In some embodiment of a compound of Formula (I), (Ia), or (Ib), the alkyl, alkenyl, alkynyl, cycloalkyl, heterocycloalkyl, aryl, and heteroaryl in R¹⁴ is optionally substituted with three R⁴.

In some embodiment of a compound of Formula (I), (Ia), or (Ib), each R⁴ is independently halogen, —CN, —OH, —OR^{a}, —NR^{c}R^{d}, —C(═O)R^{a}, —C(═O)OR^{b}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is independently optionally substituted with one, two, or three oxo, halogen, —CN, —OH, —OMe, —NH₂, C₁-C₆alkyl, C₁-C₆haloalkyl, C₁-C₆hydroxyalkyl, or C₁-C₆aminoalkyl. In some embodiment of a compound of Formula (I), (Ia), or (Ib), each R⁴ is independently oxo, halogen, —CN, —OH, —OR^{a}, —NR^{c}R^{d}, C₁-C₆alkyl, C₁-C₆haloalkyl, C₁-C₆hydroxyalkyl, or C₁-C₆aminoalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), (Ia), or (Ib), each R⁴ is independently oxo, halogen, —CN, —OH, —OR^{a}, —NR^{c}R^{d}, C₁-C₆alkyl, C₁-C₆haloalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), (Ia), or (Ib), each R⁴ is independently oxo, halogen, —CN, —OH, —OR^{a}, —NR^{c}R^{d}, C₁-C₆alkyl, C₁-C₆haloalkyl. In some embodiment of a compound of Formula (I), (Ia), or (Ib), each R⁴ is independently oxo, halogen, —CN, —OH, —OMe, —NH₂, Me, or CF₃. In some embodiment of a compound of Formula (I), (Ia), or (Ib), each R⁴ is independently halogen.

In some embodiments of a compound of Formula (I), (Ia), or (Ib), Ring B is a 6- to 8-membered heterocycloalkyl. In some embodiments of a compound of Formula (I), (Ia), or (Ib), Ring B is a 6- or 7-membered heterocycloalkyl. In some embodiments of a compound of Formula (I), (Ia), or (Ib), Ring B is a 7- or 8-membered heterocycloalkyl. In some embodiments of a compound of Formula (I), (Ia), or (Ib), Ring B is a 8- or 9-membered heterocycloalkyl. In some embodiments of a compound of Formula (I), (Ia), or (Ib), Ring B is a 6-membered heterocycloalkyl. In some embodiments of a compound of Formula (I), (Ia), or (Ib), Ring B is a 7-membered heterocycloalkyl. In some embodiments of a compound of Formula (I), (Ia), or (Ib), Ring B is a 8-membered heterocycloalkyl. In some embodiments of a compound of Formula (I), (Ia), or (Ib), Ring B is a 9-membered heterocycloalkyl.

In some embodiments of a compound of Formula (I), (Ia), or (Ib), the heterocycloalkyl of Ring B comprises 1, 2, or 3 heteroatoms selected from the group consisting of O, S, and N in addition to NR¹⁶. In some embodiments of a compound of Formula (I), (Ia), or (Ib), the heterocycloalkyl of Ring B comprises 1, 2, or 3 heteroatoms selected from the group consisting of O and N in addition to NR¹⁶. In some embodiments of a compound of Formula (I), (Ia), or (Ib), the heterocycloalkyl of Ring B comprises 1 or 2 heteroatoms selected from the group consisting of O and N in addition to NR¹⁶. In some embodiments of a compound of Formula (I), (Ia), or (Ib), the heterocycloalkyl of Ring B comprises 1, 2, or 3 heteroatoms selected from the group consisting of O and S in addition to NR¹⁶. In some embodiments of a compound of Formula (I), (Ia), or (Ib), the heterocycloalkyl of Ring B comprises 1, 2, or 3 heteroatoms selected from the group consisting of O and N in addition to NR¹⁶. In some embodiments of a compound of Formula (I), (Ia), or (Ib), the heterocycloalkyl of Ring B comprises 1, 2, or 3 heteroatoms that are O in addition to NR¹⁶. In some embodiments of a compound of Formula (I), (Ia), or (Ib), the heterocycloalkyl of Ring B comprises 1 or 2 heteroatoms that are O in addition to NR¹⁶. In some embodiments of a compound of Formula (I), (Ia), or (Ib), the heterocycloalkyl of Ring B comprises 1 heteroatom that is O in addition to NR¹⁶. In some embodiments of a compound of Formula (I), (Ia), or (Ib), the heterocycloalkyl of Ring B comprises 1, 2, or 3 heteroatoms that are N in addition to NR¹⁶. In some embodiments of a compound of Formula (I), (Ia), or (Ib), the heterocycloalkyl of Ring B comprises 1 or 2 heteroatoms that are N in addition to NR¹⁶. In some embodiments of a compound of Formula (I), (Ia), or (Ib), the heterocycloalkyl of Ring B comprises 1 heteroatom that is N in addition to NR¹⁶. In some embodiments of a compound of Formula (I), (Ia), or (Ib), the heterocycloalkyl of Ring B comprises 1, 2, or 3 heteroatoms selected from the group consisting of O, S, and N. In some embodiments of a compound of Formula (I), (Ia), or (Ib), the heterocycloalkyl of Ring B comprises 1, 2, or 3 heteroatoms selected from the group consisting of O and N. In some embodiments of a compound of Formula (I), (Ia), or (Ib), the heterocycloalkyl of Ring B comprises 1 or 2 heteroatoms selected from the group consisting of O and N.

In some embodiments of a compound of Formula (I), (Ia), or (Ib),

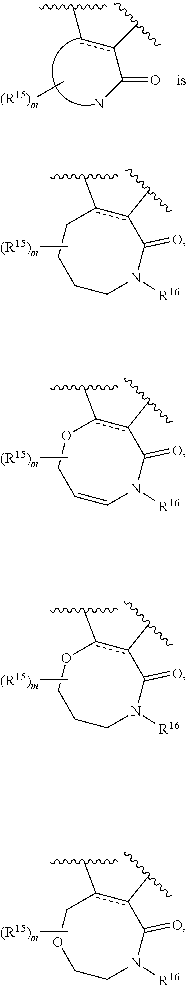

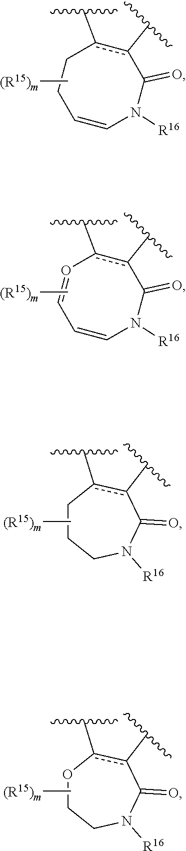

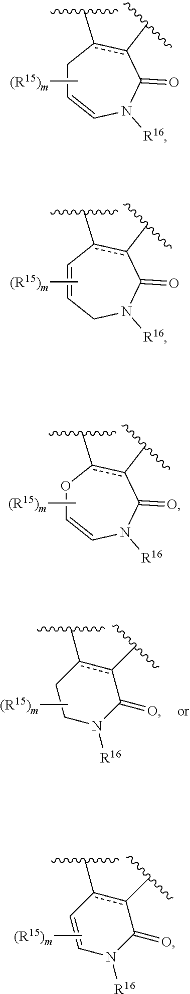

wherein [symbol] is a single bond or a double bond.

In some embodiments of a compound of Formula (I), (Ia), or (Ib),

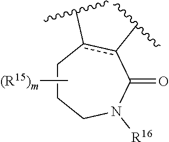

wherein [symbol] is a single bond or a double bond.

In some embodiments of a compound of Formula (I), (Ia), or (Ib), each R¹⁵ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, cycloalkyl, heterocycloalkyl, aryl, and heteroaryl is independently optionally substituted with one, two, or three R⁵. In some embodiments of a compound of Formula (I), (Ia), or (Ib), each R¹⁵ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, or C₁-C₆aminoalkyl. In some embodiments of a compound of Formula (I), (Ia), or (Ib), each R¹⁵ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, or C₁-C₆haloalkyl. In some embodiments of a compound of Formula (I), (Ia), or (Ib), each R¹⁵ is independently C₁-C₆alkyl, C₁-C₆haloalkyl, C₁-C₆hydroxyalkyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, cycloalkyl, heterocycloalkyl, aryl, and heteroaryl is independently optionally substituted with one, two, or three R⁵. In some embodiments of a compound of Formula (I), (Ia), or (Ib), each R¹⁵ is independently C₁-C₆alkyl, C₁-C₆haloalkyl, or C₁-C₆hydroxyalkyl. In some embodiments of a compound of Formula (I), (Ia), or (Ib), each R¹⁵ is independently C₁-C₆alkyl or C₁-C₆hydroxyalkyl. In some embodiments of a compound of Formula (I), (Ia), or (Ib), each R¹⁵ is independently C₁-C₆alkyl.

In some embodiment of a compound of Formula (I), (Ia), or (Ib), each alkyl, alkenyl, alkynyl, cycloalkyl, heterocycloalkyl, aryl, and heteroaryl in R¹⁵ is independently optionally substituted with one, two, or three R⁵. In some embodiment of a compound of Formula (I), (Ia), or (Ib), each alkyl, alkenyl, alkynyl, cycloalkyl, heterocycloalkyl, aryl, and heteroaryl in R¹⁵ is independently optionally substituted with one or two R⁵. In some embodiment of a compound of Formula (I), (Ia), or (Ib), each alkyl, alkenyl, alkynyl, cycloalkyl, heterocycloalkyl, aryl, and heteroaryl in R¹⁵ is independently optionally substituted with one R₅. In some embodiment of a compound of Formula (I), (Ia), or (Ib), each alkyl, alkenyl, alkynyl, cycloalkyl, heterocycloalkyl, aryl, and heteroaryl in R¹⁵ is independently optionally substituted with two R₅. In some embodiment of a compound of Formula (I), (Ia), or (Ib), each alkyl, alkenyl, alkynyl, cycloalkyl, heterocycloalkyl, aryl, and heteroaryl in R¹⁵ is independently optionally substituted with three R⁵.

In some embodiment of a compound of Formula (I), (Ia), or (Ib), each R is independently halogen, —CN, —OH, —OR^{a}, —NR^{c}R^{d}, —C(═O)R^{a}, —C(═O)OR^{b}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is independently optionally substituted with one, two, or three oxo, halogen, —CN, —OH, —OMe, —NH₂, C₁-C₆alkyl, C₁-C₆haloalkyl, C₁-C₆hydroxyalkyl, or C₁-C₆aminoalkyl. In some embodiment of a compound of Formula (I), (Ia), or (Ib), each R⁵ is independently oxo, halogen, —CN, —OH, —OR^{a}, —NR^{c}R^{d}, C₁-C₆alkyl, C₁-C₆haloalkyl, C₁-C₆hydroxyalkyl, or C₁-C₆aminoalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), (Ia), or (Ib), each R⁵ is independently oxo, halogen, —CN, —OH, —OR^{a}, —NR^{c}R^{d}, C₁-C₆alkyl, C₁-C₆haloalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), (Ia), or (Ib), each R⁵ is independently oxo, halogen, —CN, —OH, —OR^{a}, —NR^{c}R^{d}, C₁-C₆alkyl, C₁-C₆haloalkyl. In some embodiment of a compound of Formula (I), (Ia), or (Ib), each R⁵ is independently oxo, halogen, —CN, —OH, —OMe, —NH₂, Me, or CF₃. In some embodiment of a compound of Formula (I), (Ia), or (Ib), each R is independently halogen.

In some embodiments of a compound of Formula (I), (Ia), or (Ib), two R¹⁵ on the same carbon atom are taken together to form an oxo, a cycloalkyl, or a heterocycloalkyl; wherein the cycloalkyl and heterocycloalkyl is optionally substituted with one, two, or three R⁶. In some embodiments of a compound of Formula (I), (Ia), or (Ib), two R¹⁵ on the same carbon atom are taken together to form a cycloalkyl optionally substituted with one, two, or three R⁶.

In some embodiment of a compound of Formula (I), (Ia), or (Ib), the cycloalkyl and heterocycloalkyl formed when two R¹⁵ are taken together is optionally substituted with one, two, or three R⁶. In some embodiment of a compound of Formula (I), (Ia), or (Ib), the cycloalkyl and heterocycloalkyl formed when two R¹⁵ are taken together is optionally substituted with one or two R⁶. In some embodiment of a compound of Formula (I), (Ia), or (Ib), the cycloalkyl and heterocycloalkyl formed when two R¹⁵ are taken together is optionally substituted with one R⁶. In some embodiment of a compound of Formula (I), (Ia), or (Ib), the cycloalkyl and heterocycloalkyl formed when two R¹⁵ are taken together is optionally substituted with two R⁶. In some embodiment of a compound of Formula (I), (Ia), or (Ib), the cycloalkyl and heterocycloalkyl formed when two R¹⁵ are taken together is optionally substituted with three R⁶.

In some embodiment of a compound of Formula (I), (Ia), or (Ib), each R⁶ is independently halogen, —CN, —OH, —OR^{a}, —NR^{c}R^{d}, —C(═O)R^{a}, —C(═O)OR^{b}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is independently optionally substituted with one, two, or three oxo, halogen, —CN, —OH, —OMe, —NH₂, C₁-C₆alkyl, C₁-C₆haloalkyl, C₁-C₆hydroxyalkyl, or C₁-C₆aminoalkyl. In some embodiment of a compound of Formula (I), (Ia), or (Ib), each R⁶ is independently oxo, halogen, —CN, —OH, —OR^{a}, —NR^{c}R^{d}, C₁-C₆alkyl, C₁-C₆haloalkyl, C₁-C₆hydroxyalkyl, or C₁-C₆aminoalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), (Ia), or (Ib), each R⁶ is independently oxo, halogen, —CN, —OH, —OR^{a}, —NR^{c}R^{d}, C₁-C₆alkyl, C₁-C₆haloalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), (Ia), or (Ib), each R⁶ is independently oxo, halogen, —CN, —OH, —OR^{a}, —NR^{c}R^{d}, C₁-C₆alkyl, C₁-C₆haloalkyl. In some embodiment of a compound of Formula (I), (Ia), or (Ib), each R⁶ is independently oxo, halogen, —CN, —OH, —OMe, —NH₂, Me, or CF₃. In some embodiment of a compound of Formula (I), (Ia), or (Ib), each R⁶ is independently halogen.

In some embodiments of a compound of Formula (I), (Ia), or (Ib), two R¹⁵ on adjacent atoms are taken together to form a cycloalkyl or a heterocycloalkyl; wherein the cycloalkyl and heterocycloalkyl is optionally substituted with one, two, or three R⁷. In some embodiments of a compound of Formula (I), (Ia), or (Ib), two R¹⁵ on adjacent atoms are taken together to form a heterocycloalkyl optionally substituted with one, two, or three R⁷.

In some embodiment of a compound of Formula (I), (Ia), or (Ib), the cycloalkyl and heterocycloalkyl formed when two R¹⁵ are taken together is optionally substituted with one, two, or three R⁷. In some embodiment of a compound of Formula (I), (Ia), or (Ib), the cycloalkyl and heterocycloalkyl formed when two R¹⁵ are taken together is optionally substituted with one or two R⁷. In some embodiment of a compound of Formula (I), (Ia), or (Ib), the cycloalkyl and heterocycloalkyl formed when two R¹⁵ are taken together is optionally substituted with one R⁷. In some embodiment of a compound of Formula (I), (Ia), or (Ib), the cycloalkyl and heterocycloalkyl formed when two R¹⁵ are taken together is optionally substituted with two R⁷. In some embodiment of a compound of Formula (I), (Ia), or (Ib), the cycloalkyl and heterocycloalkyl formed when two R¹⁵ are taken together is optionally substituted with three R⁷.

In some embodiment of a compound of Formula (I), (Ia), or (Ib), each R⁷ is independently halogen, —CN, —OH, —OR^{a}, —NR^{c}R^{d}, —C(═O)R^{a}, —C(═O)OR^{b}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is independently optionally substituted with one, two, or three oxo, halogen, —CN, —OH, —OMe, —NH₂, C₁-C₆alkyl, C₁-C₆haloalkyl, C₁-C₆hydroxyalkyl, or C₁-C₆aminoalkyl. In some embodiment of a compound of Formula (I), (Ia), or (Ib), each R⁷ is independently oxo, halogen, —CN, —OH, —OR^{a}, —NR^{c}R^{d}, C₁-C₆alkyl, C₁-C₆haloalkyl, C₁-C₆hydroxyalkyl, or C₁-C₆aminoalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), (Ia), or (Ib), each R⁷ is independently oxo, halogen, —CN, —OH, —OR^{a}, —NR^{c}R^{d}, C₁-C₆alkyl, C₁-C₆haloalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), (Ia), or (Ib), each R⁷ is independently oxo, halogen, —CN, —OH, —OR^{a}, —NR^{c}R^{d}, C₁-C₆alkyl, C₁-C₆haloalkyl. In some embodiment of a compound of Formula (I), (Ia), or (Ib), each R⁷ is independently oxo, halogen, —CN, —OH, —OMe, —NH₂, Me, or CF₃. In some embodiment of a compound of Formula (I), (Ia), or (Ib), each R⁷ is independently halogen.

In some embodiments of a compound of Formula (I), (Ia), or (Ib), two R¹⁵ on adjacent atoms are taken together to form a double bond.

In some embodiments of a compound of Formula (I), (Ia), or (Ib), m is 0-4. In some embodiments of a compound of Formula (I), (Ia), or (Ib), m is 0-5. In some embodiments of a compound of Formula (I), (Ia), or (Ib), m is 2-6. In some embodiments of a compound of Formula (I), (Ia), or (Ib), m is 2-5. In some embodiments of a compound of Formula (I), (Ia), or (Ib), m is 2-4. In some embodiments of a compound of Formula (I), (Ia), or (Ib), m is 0-2. In some embodiments of a compound of Formula (I), (Ia), or (Ib), m is 0 or 1. In some embodiments of a compound of Formula (I), (Ia), or (Ib), m is 0-3. In some embodiments of a compound of Formula (I), (Ia), or (Ib), m is 1-3. In some embodiments of a compound of Formula (I), (Ia), or (Ib), m is 1 or 2. In some embodiments of a compound of Formula (I), (Ia), or (Ib), m is 0. In some embodiments of a compound of Formula (I), (Ia), or (Ib), m is 1. In some embodiments of a compound of Formula (I), (Ia), or (Ib), m is 2. In some embodiments of a compound of Formula (I), (Ia), or (Ib), m is 3. In some embodiments of a compound of Formula (I), (Ia), or (Ib), m is 4. In some embodiments of a compound of Formula (I), (Ia), or (Ib), m is 5. In some embodiments of a compound of Formula (I), (Ia), or (Ib), m is 6.

In some embodiments of a compound of Formula (I), (Ia), or (Ib), R¹⁶ is hydrogen.

In some embodiments of a compound of Formula (I), (Ia), or (Ib), or a pharmaceutically acceptable salt, solvate, or stereoisomer thereof, each R^{a} is independently C₁-C₆alkyl, C₁-C₆haloalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), or a pharmaceutically acceptable salt, solvate, or stereoisomer thereof, each R^{a} is independently C₁-C₆alkyl, C₁-C₆haloalkyl, cycloalkyl, or heterocycloalkyl. In some embodiments of a compound of Formula (I), (Ia), or (Ib), or a pharmaceutically acceptable salt, solvate, or stereoisomer thereof, each R^{a} is independently C₁-C₆alkyl, C₁-C₆haloalkyl, or cycloalkyl. In some embodiments of a compound of Formula (I), (Ia), or (Ib), or a pharmaceutically acceptable salt, solvate, or stereoisomer thereof, each R^{a} is independently C₁-C₆alkyl or C₁-C₆haloalkyl. In some embodiments of a compound of Formula (I), (Ia), or (Ib), or a pharmaceutically acceptable salt, solvate, or stereoisomer thereof, each R^{a} is independently C₁-C₆alkyl.

In some embodiments of a compound of Formula (I), (Ia), or (Ib), or a pharmaceutically acceptable salt, solvate, or stereoisomer thereof, each R^{b} is independently hydrogen, C₁-C₆alkyl, C₁-C₆haloalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), or a pharmaceutically acceptable salt, solvate, or stereoisomer thereof, each R^{b} is independently hydrogen, C₁-C₆alkyl, C₁-C₆haloalkyl, cycloalkyl, or heterocycloalkyl. In some embodiments of a compound of Formula (I), (Ia), or (Ib), or a pharmaceutically acceptable salt, solvate, or stereoisomer thereof, each R^{b} is independently hydrogen, C₁-C₆alkyl, C₁-C₆haloalkyl, or cycloalkyl. In some embodiments of a compound of Formula (I), (Ia), or (Ib), or a pharmaceutically acceptable salt, solvate, or stereoisomer thereof, each R^{b} is independently hydrogen, C₁-C₆alkyl, or C₁-C₆haloalkyl. In some embodiments of a compound of Formula (I), (Ia), or (Ib), or a pharmaceutically acceptable salt, solvate, or stereoisomer thereof, each R^{b} is independently hydrogen or C₁-C₆alkyl. In some embodiments of a compound of Formula (I), (Ia), or (Ib), or a pharmaceutically acceptable salt, solvate, or stereoisomer thereof, each R^{b} is hydrogen. In some embodiments of a compound of Formula (I), (Ia), or (Ib), or a pharmaceutically acceptable salt, solvate, or stereoisomer thereof, each R^{b} is independently C₁-C₆alkyl.

In some embodiments of a compound of Formula (I), (Ia), or (Ib), or a pharmaceutically acceptable salt, solvate, or stereoisomer thereof, each R^{c} and R^{d} is independently hydrogen, C₁-C₆alkyl, C₁-C₆haloalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), or a pharmaceutically acceptable salt, solvate, or stereoisomer thereof, each R^{c} and R^{d} is independently hydrogen, C₁-C₆alkyl, C₁-C₆haloalkyl, cycloalkyl, or heterocycloalkyl. In some embodiments of a compound of Formula (I), (Ia), or (Ib), or a pharmaceutically acceptable salt, solvate, or stereoisomer thereof, each R^{c} and R^{d} is independently hydrogen, C₁-C₆alkyl, C₁-C₆haloalkyl, or cycloalkyl. In some embodiments of a compound of Formula (I), (Ia), or (Ib), or a pharmaceutically acceptable salt, solvate, or stereoisomer thereof, each R^{c} and R^{d} is independently hydrogen, C₁-C₆alkyl, or C₁-C₆haloalkyl. In some embodiments of a compound of Formula (I), (Ia), or (Ib), or a pharmaceutically acceptable salt, solvate, or stereoisomer thereof, each R^{c} and R^{d} is independently hydrogen or C₁-C₆alkyl. In some embodiments of a compound of Formula (I), (Ia), or (Ib), or a pharmaceutically acceptable salt, solvate, or stereoisomer thereof, each R^{c} and R^{d} is hydrogen.

In some embodiments of a compound of Formula (I), (Ia), or (Ib), or a pharmaceutically acceptable salt, solvate, or stereoisomer thereof, R^{c} and R^{d} are taken together with the atom to which they are attached to form a heterocycloalkyl optionally substituted with one, two, or three halogen, C₁-C₆alkyl, or C₁-C₆haloalkyl.

Any combination of the groups described above for the various variables is contemplated herein. Throughout the specification, groups and substituents thereof are chosen by one skilled in the field to provide stable moieties and compounds.

Described herein is a compound of Formula (I), (Ia), or (Ib), or a pharmaceutically acceptable salt, solvate, or stereoisomer thereof, selected from a compound in Table 1.

TABLE 1
Exemplary compounds

| | | | ESI-MS |
|---|---|---|---|
| Ex. | Structure | Chemical Name | (M + H)⁺ (m/z) |
| 1 | 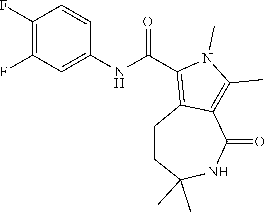 [SMILES: Cc1c2c(c(C(=O)Nc3ccc(F)c(F)c3)n1C)CCC(C)(C)NC2=O] | N-(3,4-difluorophenyl)-1,2,6,6- tetramethyl-8-oxo-1,4,5,6,7,8- hexahydropyrrolo[2,3-c]azepine-3- carboxamide | 362.1 |
| 2 | 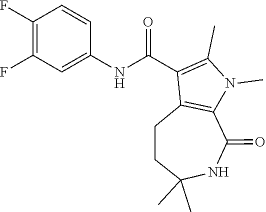 [SMILES: Cc1c(C(=O)Nc2ccc(F)c(F)c2)c2c(n1C)C(=O)NC(C)(C)CC2] | N-(3,4-difluorophenyl)-1,2,6,6- tetramethyl-8-oxo-1,4,5,6,7,8- hexahydropyrrolo[2,3-c]azepine-3- carboxamide | 362.1 |
| 3 | 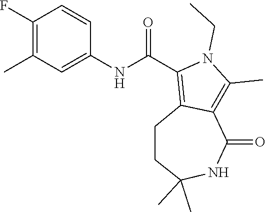 [SMILES: CCn1c(C)c2c(c1C(=O)Nc1ccc(F)c(C)c1)CCC(C)(C)NC2=O] | 2-ethyl-N-(4-fluoro-3- methylphenyl)-3,6,6-trimethyl-4- oxo-2,4,5,6,7,8- hexahydropyrrolo[3,4-c]azepine-1- carboxamide | 372.1 |
| 4 | 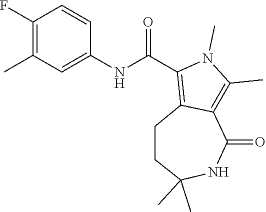 [SMILES: Cc1cc(NC(=O)c2c3c(c(C)n2C)C(=O)NC(C)(C)CC3)ccc1F] | N-(4-fluoro-3-methylphenyl)- 2,3,6,6-tetramethyl-4-oxo- 2,4,5,6,7,8-hexahydropyrrolo[3,4- c]azepine-1-carboxamide | 358.1 |
| 5 | 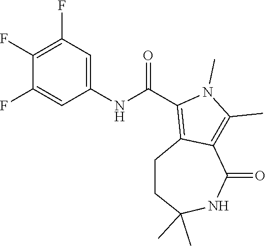 [SMILES: Cc1c2c(c(C(=O)Nc3cc(F)c(F)c(F)c3)n1C)CCC(C)(C)NC2=O] | 2,3,6,6-tetramethyl-4-oxo-N- (3,4,5-trifluorophenyl)-2,4,5,6,7,8- hexahydropyrrolo[3,4-c]azepine-1- carboxamide | 380.2 |
| 6 | 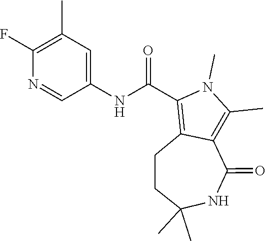 [SMILES: Cc1cc(NC(=O)c2c3c(c(C)n2C)C(=O)NC(C)(C)CC3)cnc1F] | N-(6-fluoro-5-methylpyridin-3-yl)- 2,3,6,6-tetramethyl-4-oxo- 2,4,5,6,7,8-hexahydropyrrolo[3,4- c]azepine-1-carboxamide | 359.2 |
| 7 | 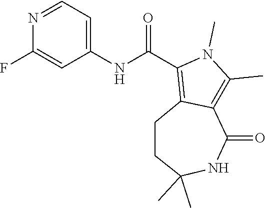 [SMILES: Cc1c2c(c(C(=O)Nc3ccnc(F)c3)n1C)CCC(C)(C)NC2=O] | N-(2-fluoropyridin-4-yl)-2,3,6,6- tetramethyl-4-oxo-2,4,5,6,7,8- hexahydropyrrolo[3,4-c]azepine-1- carboxamide | 345.1 |
| 8 | 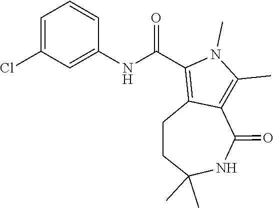 [SMILES: Cc1c2c(c(C(=O)Nc3cccc(Cl)c3)n1C)CCC(C)(C)NC2=O] | N-(3-chlorophenyl)-2,3,6,6- tetramethyl-4-oxo-2,4,5,6,7,8- hexahydropyrrolo[3,4-c]azepine-1- carboxamide | 360.1 |
| 9 | 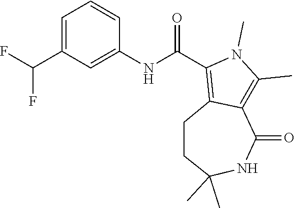 [SMILES: Cc1c2c(c(C(=O)Nc3cccc(C(F)F)c3)n1C)CCC(C)(C)NC2=O] | N-(3-(difluoromethyl)phenyl)- 2,3,6,6-tetramethyl-4-oxo- 2,4,5,6,7,8-hexahydropyrrolo[3,4- c]azepine-1-carboxamide | 376.1 |
| 10 | 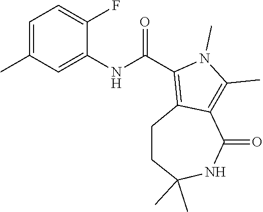 | N-(2-fluoro-5-methylphenyl)- 2,3,6,6-tetramethyl-4-oxo- 2,4,5,6,7,8-hexahydropyrrolo[3,4- c]azepine-1-carboxamide | 358.1 |
| 11 | 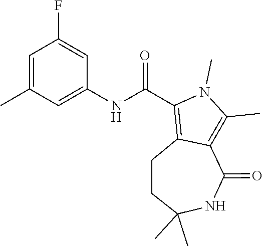 | N-(3-fluoro-5-methylphenyl)- 2,3,6,6-tetramethyl-4-oxo- 2,4,5,6,7,8-hexahydropyrrolo[3,4- c]azepine-1-carboxamide | 358.1 |
| 12 | 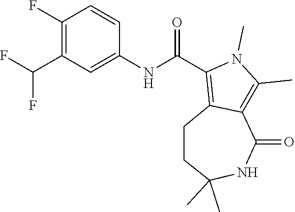 [SMILES: Cc1c2c(c(C(=O)Nc3ccc(F)c(C(F)F)c3)n1C)CCC(C)(C)NC2=O] | N-(3-(difluoromethyl)-4- fluorophenyl)-2,3,6,6-tetramethyl- 4-oxo-2,4,5,6,7,8- hexahydropyrrolo[3,4-c]azepine-1- carboxamide | 394.1 |
| 13 | 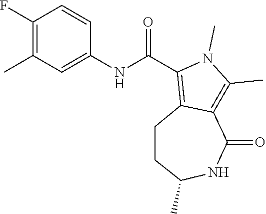 | (R)-N-(4-fluoro-3-methylphenyl)- 2,3,6-trimethyl-4-oxo-2,4,5,6,7,8- hexahydropyrrolo[3,4-c]azepine-1- carboxamide | |
| 14 | 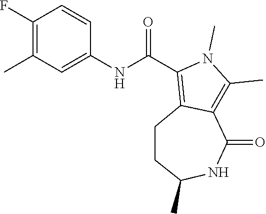 | (S)-N-(4-fluoro-3-methylphenyl)- 2,3,6-trimethyl-4-oxo-2,4,5,6,7,8- hexahydropyrrolo[3,4-c]azepine-1- carboxamide | |
| 15 | 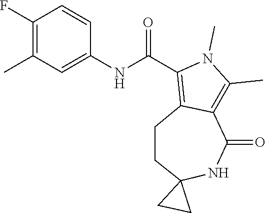 | N-(4-fluoro-3-methylphenyl)-2′,3′- dimethyl-4′-oxo-4′,5′,7′,8′- tetrahydro-2′H- spiro[cyclopropane-1,6′- pyrrolo[3,4-c]azepine]-1′- carboxamide | |
| 16 | 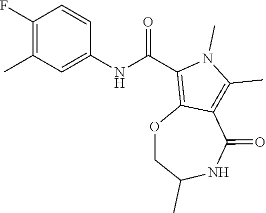 | N-(4-fluoro-3-methylphenyl)- 3,6,7-trimethyl-5-oxo-3,4,5,7- tetrahydro-2H-pyrrolo[3,4- f][1,4]oxazepine-8-carboxamide | |
| 17 | 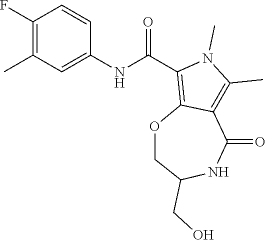 | N-(4-fluoro-3-methylphenyl)-3- (hydroxymethyl)-6,7-dimethyl-5- oxo-3,4,5,7-tetrahydro-2H- pyrrolo[3,4-f][1,4]oxazepine-8- carboxamide | |
| 18 | 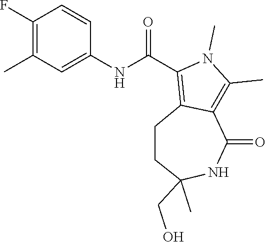 | N-(4-fluoro-3-methylphenyl)-6- (hydroxymethyl)-2,3,6-trimethyl- 4-oxo-2,4,5,6,7,8- hexahydropyrrolo[3,4-c]azepine-1- carboxamide | |
| 19 | 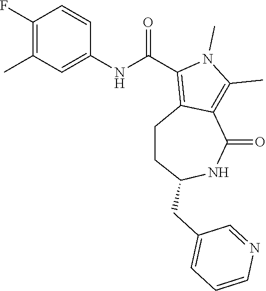 | (S)-N-(4-fluoro-3-methylphenyl)- 2,3-dimethyl-4-oxo-6-(pyridin-3- ylmethyl)-2,4,5,6,7,8- hexahydropyrrolo[3,4-c]azepine-1- carboxamide | |
| 20 | 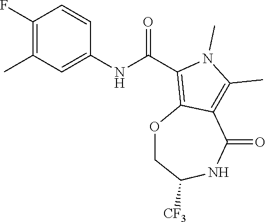 | (S)-N-(4-fluoro-3-methylphenyl)- 6,7-dimethyl-5-oxo-3- (trifluoromethyl)-3,4,5,7- tetrahydro-2H-pyrrolo[3,4- f][1,4]oxazepine-8-carboxamide | |
| 21 | 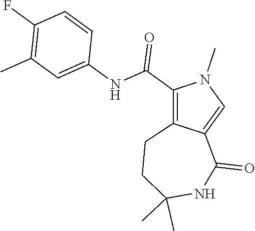 [SMILES: Cc1cc(NC(=O)c2c3c(cn2C)C(=O)NC(C)(C)CC3)ccc1F] | N-(4-fluoro-3-methylphenyl)- 2,6,6-trimethyl-4-oxo-2,4,5,6,7,8- hexahydropyrrolo[3,4-c]azepine-1- carboxamide | |
| 22 | 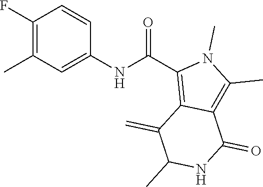 | N-(4-fluoro-3-methylphenyl)- 2,3,6-trimethyl-7-methylene-4- oxo-4,5,6,7-tetrahydro-2H- pyrrolo[3,4-c]pyridine-1- carboxamide | 342.2 |
| 23 | 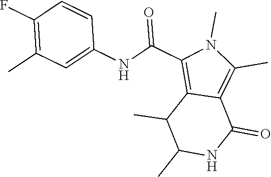 [SMILES: Cc1cc(NC(=O)c2c3c(c(C)n2C)C(=O)NC(C)C3C)ccc1F] | N-(4-fluoro-3-methylphenyl)- 2,3,6,7-tetramethyl-4-oxo-4,5,6,7- tetrahydro-2H-pyrrolo[3,4- c]pyridine-1-carboxamide | 344.2 |
| 24 | 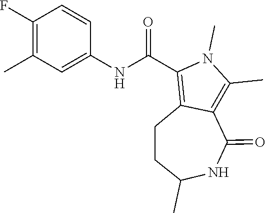 | N-(4-fluoro-3-methylphenyl)- 2,3,6-trimethyl-4-oxo-2,4,5,6,7,8- hexahydropyrrolo[3,4-c]azepine-1- carboxamide | 344.2 |
| 25 | 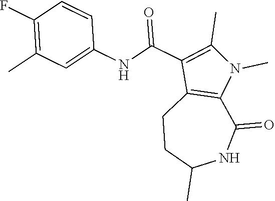 [SMILES: Cc1cc(NC(=O)c2c3c(n(C)c2C)C(=O)NC(C)CC3)ccc1F] | N-(4-fluoro-3-methylphenyl)- 1,2,6-trimethyl-8-oxo-1,4,5,6,7,8- hexahydropyrrolo[2,3-c]azepine-3- carboxamide | 344.2 |
| 26 | 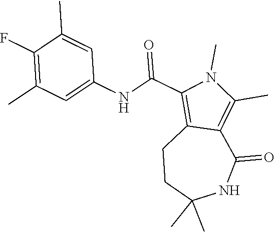 [SMILES: Cc1cc(NC(=O)c2c3c(c(C)n2C)C(=O)NC(C)(C)CC3)cc(C)c1F] | N-(4-fluoro-3,5-dimethylphenyl)- 2,3,6,6-tetramethyl-4-oxo- 2,4,5,6,7,8-hexahydropyrrolo[3,4- c]azepine-1-carboxamide | |
| 27 | 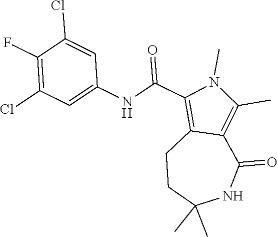 [SMILES: Cc1c2c(c(C(=O)Nc3cc(Cl)c(F)c(Cl)c3)n1C)CCC(C)(C)NC2=O] | N-(3,5-dichloro-4-fluorophenyl)- 2,3,6,6-tetramethyl-4-oxo- 2,4,5,6,7,8-hexahydropyrrolo[3,4- c]azepine-1-carboxamide | |
| 28 | 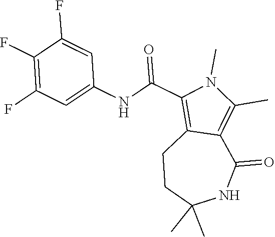 [SMILES: Cc1c2c(c(C(=O)Nc3cc(F)c(F)c(F)c3)n1C)CCC(C)(C)NC2=O] | 2,3,6,6-tetramethyl-4-oxo-N- (3,4,5-trifluorophenyl)-2,4,5,6,7,8- hexahydropyrrolo[3,4-c]azepine-1- carboxamide | |
| 29 | 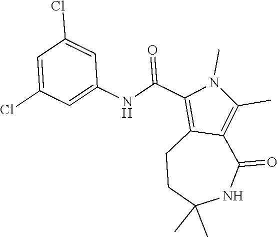 [SMILES: Cc1c2c(c(C(=O)Nc3cc(Cl)cc(Cl)c3)n1C)CCC(C)(C)NC2=O] | N-(3,5-dichlorophenyl)-2,3,6,6- tetramethyl-4-oxo-2,4,5,6,7,8- hexahydropyrrolo[3,4-c]azepine-1- carboxamide | |
| 30 | 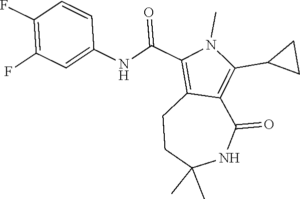 [SMILES: Cn1c(C(=O)Nc2ccc(F)c(F)c2)c2c(c1C1CC1)C(=O)NC(C)(C)CC2] | 3-cyclopropyl-N-(3,4- difluorophenyl)-2,6,6-trimethyl-4- oxo-2,4,5,6,7,8- hexahydropyrrolo[3,4-c]azepine-1- carboxamide | |
| 31 | 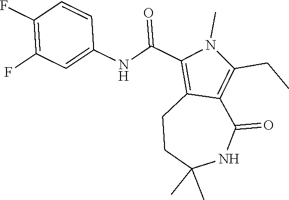 [SMILES: CCc1c2c(c(C(=O)Nc3ccc(F)c(F)c3)n1C)CCC(C)(C)NC2=O] | N-(3,4-difluorophenyl)-3-ethyl- 2,6,6-trimethyl-4-oxo-2,4,5,6,7,8- hexahydropyrrolo[3,4-c]azepine-1- carboxamide | |
| 32 | 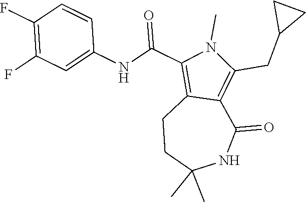 [SMILES: Cn1c(CC2CC2)c2c(c1C(=O)Nc1ccc(F)c(F)c1)CCC(C)(C)NC2=O] | 3-(cyclopropylmethyl)-N-(3,4- difluorophenyl)-2,6,6-trimethyl-4- oxo-2,4,5,6,7,8- hexahydropyrrolo[3,4-c]azepine-1- carboxamide | |
| 33 | 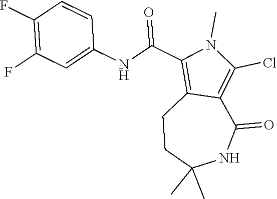 [SMILES: Cn1c(Cl)c2c(c1C(=O)Nc1ccc(F)c(F)c1)CCC(C)(C)NC2=O] | 3-chloro-N-(3,4-difluorophenyl)- 2,6,6-trimethyl-4-oxo-2,4,5,6,7,8- hexahydropyrrolo[3,4-c]azepine-1- carboxamide | |
| 34 | 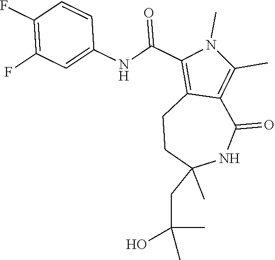 [SMILES: Cc1c2c(c(C(=O)Nc3ccc(F)c(F)c3)n1C)CCC(C)(CC(C)(C)O)NC2=O] | N-(3,4-difluorophenyl)-6-(2- hydroxy-2-methylpropyl)-2,3,6- trimethyl-4-oxo-2,4,5,6,7,8- hexahydropyrrolo[3,4-c]azepine-1- carboxamide | |
| 35 | 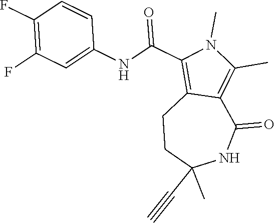 [SMILES: C#CC1(C)CCc2c(c(C)n(C)c2C(=O)Nc2ccc(F)c(F)c2)C(=O)N1] | N-(3,4-difluorophenyl)-6-ethynyl- 2,3,6-trimethyl-4-oxo-2,4,5,6,7,8- hexahydropyrrolo[3,4-c]azepine-1- carboxamide | |
| 36 | 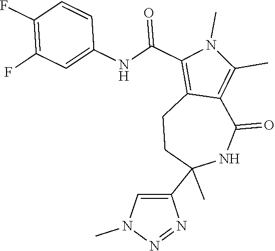 [SMILES: Cc1c2c(c(C(=O)Nc3ccc(F)c(F)c3)n1C)CCC(C)(c1cn(C)nn1)NC2=O] | N-(3,4-difluorophenyl)-2,3,6- trimethyl-6-(1-methyl-1H-1,2,3- triazol-4-yl)-4-oxo-2,4,5,6,7,8- hexahydropyrrolo[3,4-c]azepine-1- carboxamide | |
| 37 | 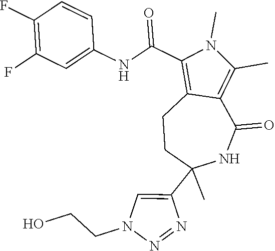 [SMILES: Cc1c2c(c(C(=O)Nc3ccc(F)c(F)c3)n1C)CCC(C)(c1cn(CCO)nn1)NC2=O] | N-(3,4-difluorophenyl)-6-(l-(2- hydroxyethyl)-1H-1,2,3-triazol-4- yl)-2,3,6-trimethyl-4-oxo- 2,4,5,6,7,8-hexahydropyrrolo[3,4- c]azepine-1-carboxamide | |
| 38 | 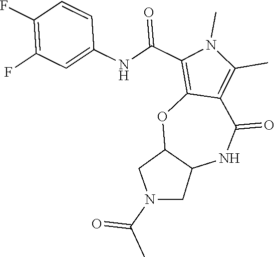 [SMILES: CC(=O)N1CC2NC(=O)c3c(c(C(=O)Nc4ccc(F)c(F)c4)n(C)c3C)OC2C1] | 6-acetyl-N-(3,4-difluorophenyl)- 1,2-dimethyl-9-oxo- 2,4a,5,6,7,7a,8,9- octahydrodipyrrolo[3,4-b:3′,4′- f][1,4]oxazepine-3-carboxamide | |
| 39 | 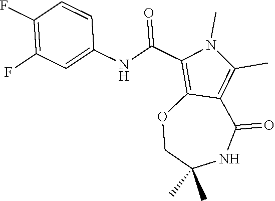 | N-(3,4-difluorophenyl)-3,3,6,7- tetramethyl-5-oxo-3,4,5,7- tetrahydro-2H-pyrrolo[3,4- f][1,4]oxazepine-8-carboxamide | |
| 40 | 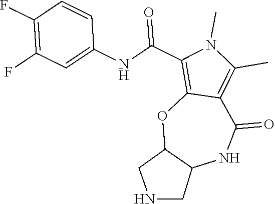 [SMILES: Cc1c2c(c(C(=O)Nc3ccc(F)c(F)c3)n1C)OC1CNCC1NC2=O] | N-(3,4-difluorophenyl)-1,2- dimethyl-9-oxo-2,4a,5,6,7,7a,8,9- octahydrodipyrrolo[3,4-b:3′,4′- f][1,4]oxazepine-3-carboxamide | |
| 41 | 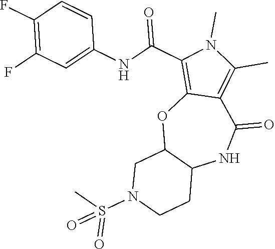 [SMILES: Cc1c2c(c(C(=O)Nc3ccc(F)c(F)c3)n1C)OC1CN(S(C)(=O)=O)CCC1NC2=O] | N-(3,4-difluorophenyl)-1,2- dimethyl-6-(methylsulfonyl)-10- oxo-4a,5,6,7,8,8a,9,10-octahydro- 2H-pyrido[3,4-b]pyrrolo[3,4- f][1,4]oxazepine-3-carboxamide | |
| 42 | 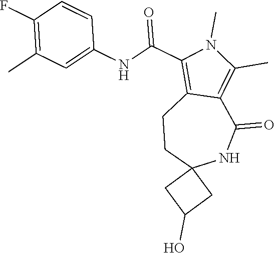 [SMILES: Cc1cc(NC(=O)c2c3c(c(C)n2C)C(=O)NC2(CC3)CC(O)C2)ccc1F] | N-(4-fluoro-3-methylphenyl)-3- hydroxy-2′,3′-dimethyl-4′-oxo- 4′,5′,7′,8′-tetrahydro-2′H- spiro[cyclobutane-1,6′-pyrrolo[3,4- c]azepine]-1′-carboxamide | |
| 43 | 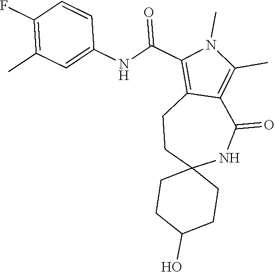 [SMILES: Cc1cc(NC(=O)c2c3c(c(C)n2C)C(=O)NC2(CC3)CCC(O)CC2)ccc1F] | N-(4-fluoro-3-methylphenyl)-4- hydroxy-2′,3′-dimethyl-4′-oxo- 4′,5′,7′,8′-tetrahydro-2′H- spiro[cyclohexane-1,6′- pyrrolo[3,4-c]azepine]-1′- carboxamide | |
| 44 | 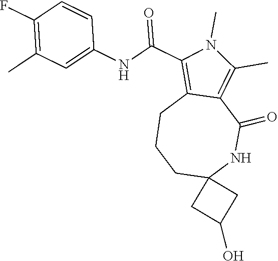 [SMILES: Cc1cc(NC(=O)c2c3c(c(C)n2C)C(=O)NC2(CCC3)CC(O)C2)ccc1F] | N-(4-fluoro-3-methylphenyl)-3- hydroxy-2′,3′-dimethyl-4′-oxo- 2′,4′,5′,7′,8′,9′- hexahydrospiro[cyclobutane-1,6′- pyrrolo[3,4-c]azocine]-1′- carboxamide | |
| 45 | 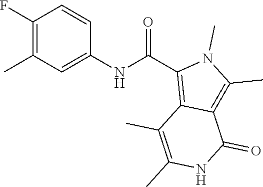 | N-(4-fluoro-3-methylphenyl)- 2,3,6,7-tetramethyl-4-oxo-4,5- dihydro-2H-pyrrolo[3,4- c]pyridine-1-carboxamide | 342.2 |
| 46 | 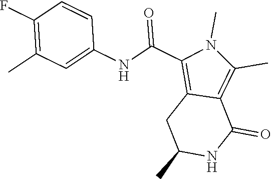 [SMILES: Cc1cc(NC(=O)c2c3c(c(C)n2C)C(=O)N[C@@H](C)C3)ccc1F] | (S)-N-(4-fluoro-3-methylphenyl)- 2,3,6-trimethyl-4-oxo-4,5,6,7- tetrahydro-2H-pyrrolo[3,4- c]pyridine-1-carboxamide | 330.1 |
| 47 | 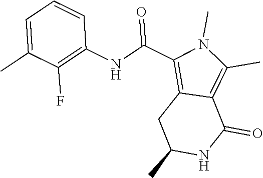 | (S)-N-(2-fluoro-3-methylphenyl)- 2,3,6-trimethyl-4-oxo-4,5,6,7- tetrahydro-2H-pyrrolo[3,4- c]pyridine-1-carboxamide | 330.1 |
| 48 | 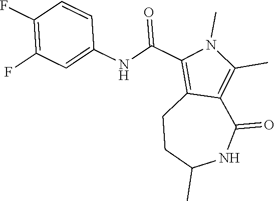 [SMILES: Cc1c2c(c(C(=O)Nc3ccc(F)c(F)c3)n1C)CCC(C)NC2=O] | N-(3,4-difluorophenyl)-2,3,6- trimethyl-4-oxo-2,4,5,6,7,8- hexahydropyrrolo[3,4-c]azepine-1- carboxamide | 348.1 |

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Method of Treatment

The compounds described herein find use in a variety of applications for human and animal health. In some embodiments, the compounds described herein are inhibitors of hepatitis B virus (HBV).

In some embodiments, the compounds described herein are capsid inhibitor. In some embodiments, the compounds described herein are used in treating HBV infection and related conditions, including chronic hepatitis B, HBV/HDV co-infection, HBV/HCV co-infection, HBV/HIV co-infection, inflammation, necrosis, cirrhosis, hepatocellular carcinoma, hepatic decompensation and hepatic injury from an HBV infection.

In some embodiments, the efficacy of treatment is determined using quantification of viral load or other evidence of infection, such as through measurement of HBeAg (hepatitis B c-antigen), HBsAg, HBV DNA levels, ALT (Alanine Transaminase) activity levels, serum HBV levels, and the like, thereby allowing adjustment of treatment dose, treatment frequency, and treatment length.

In some embodiments, the compounds described herein reduce viral load in an individual suffering from an HBV infection.

### Pharmaceutical Compositions/Formulations

The compounds described herein are administered to a subject in need thereof, either alone or in combination with pharmaceutically acceptable carriers, excipients or diluents, in a pharmaceutical composition, according to standard pharmaceutical practice. In one embodiment, the compounds of this invention may be administered to animals. The compounds can be administered orally or parenterally, including the intravenous, intramuscular, intraperitoneal, subcutaneous, rectal and topical routes of administration.

In another aspect, provided herein are pharmaceutical compositions comprising a compound describe herein, or a pharmaceutically acceptable salt, solvate, or stereoisomer thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical compositions described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

Pharmaceutical compositions including compounds described herein, or a pharmaceutically acceptable salt, solvate, or stereoisomer thereof are manufactured in a conventional manner, such as, by way of example only, by means of conventional mixing, dissolving, granulating, dragee-making, levigating, emulsifying, encapsulating, entrapping, or compression processes.

Pharmaceutical compositions for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

Pharmaceutical compositions that are administered orally include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added.

Pharmaceutical compositions for parental use are formulated as infusions or injections. In some embodiments, the pharmaceutical composition suitable for injection or infusion includes sterile aqueous solutions, or dispersions, or sterile powders comprising a compound described herein, or a pharmaceutically acceptable salt, solvate, or stereoisomer thereof. In some embodiments, the pharmaceutical composition comprises a liquid carrier. In some embodiments, the liquid carrier is a solvent or liquid dispersion medium comprising, for example, water, saline, ethanol, a polyol (for example, glycerol, propylene glycol, liquid polyethylene glycols, and the like), vegetable oils, nontoxic glyceryl esters, and any combinations thereof. In some embodiments, the pharmaceutical compositions further comprise a preservative to prevent growth of microorganisms.

### Combination

Disclosed herein are methods of treating hepatitis B using a compound disclosed herein in combination with additional therapeutic agents useful for treating chronic HBV infection.

In some embodiments, the compound disclosed herein in combination with additional therapeutic agents useful for treating an HBV infection are administered simultaneously. In some embodiments, the compound disclosed herein in combination with additional therapeutic agents useful for treating an HBV infection are administered sequentially.

Non-limiting examples of additional therapeutic agents useful for treating HBV infections include: reverse transcriptase inhibitors; HBV polymerase inhibitors, capsid inhibitors; cccDNA formation inhibitors; RNA destabilizers; checkpoint inhibitors (e.g., PD-1/PD-L1 inhibitors); therapeutic vaccines; RNA interference (RNAi) therapeutics; antisense-based therapeutics, HBV entry inhibitors; TLR agonists; RIG-I agonists; or interferons.

### Reverse Transcriptase Inhibitors

In some embodiments, the compound described herein is used in combination with a reverse transcriptase inhibitor. In certain embodiments, the reverse transcriptase inhibitor is a reverse transcriptase inhibitor (NARTI or NRTI). In other embodiments, the reverse transcriptase inhibitor is a nucleotide analog reverse transcriptase inhibitor (NtARTI or NtRTI). Reverse transcriptase inhibitors include, but are not limited to, entecavir, clevudine, telbivudine, lamivudine, adefovir, and tenofovir, tenofovir disoproxil, tenofovir alafenamide, adefovir dipovoxil, or any combination thereof. In some embodiments, the compound described herein is used in combination with tenofovir. In some embodiments, the compound described herein is used in combination with entecavir.

### HBV Polymerase Inhibitors

In some embodiments, the compound described herein is used in combination with an HBV polymerase inhibitor. In some embodiments, the HBV polymerase inhibitor is entecavir, lamivudine, telbivudine, adefovir, tenofovir disoproxil fumarate, tenofovir alafenamide fumarate (TAF), tenofovir disoproxil orotate, or tenofovir disopropxil aspartate.

### Capsid Inhibitors

In some embodiments, the compound described herein is used in combination with a capsid inhibitor. For example, a capsid assembly inhibitor includes, but is not limited to, any compound that inhibits capsid assembly, induces formation of non-capsid polymers, promotes excess capsid assembly or misdirected capsid assembly, affects capsid stabilization, and/or inhibits encapsidation of RNA (pgRNA). Capsid inhibitors also include any compound that inhibits capsid function in a downstream event(s) within the replication process (e.g., viral DNA synthesis, transport of relaxed circular DNA (rcDNA) into the nucleus, covalently closed circular DNA (cccDNA) formation, virus maturation, budding and/or release, and the like). For example, in certain embodiments, the inhibitor detectably inhibits the expression level or biological activity of the capsid protein as measured, e.g., using an assay described herein. In some embodiments, the capsid inhibitor is NVR 3-778, GLS-4, AB-423, AB-506, JNJ-56136379, JNJ-64530440, ABI-H0731, ABI-H2158, ABI-H3733, EDP-514, GLP-26, ALG-000184, ALG-001024, ALG-001075, QL-007, QL-OA6a, CB-HBV-001, and RO7049389.

cccDNA Formation Inhibitors (cccDNA)

In some embodiments, the compound described herein is used in combination with a covalently closed circular DNA (cccDNA). Covalently closed circular DNA (cccDNA) is generated in the cell nucleus from viral rcDNA and serves as the transcription template for viral mRNAs. In some embodiments, the cccDNA formation inhibitor includes compounds that are capable of inhibiting the formation and/or stability of cccDNA either directly or indirectly. In some embodiments, a cccDNA formation inhibitor includes, but is not limited to, any compound that inhibits capsid disassembly, rcDNA entry into the nucleus, and/or the conversion of rcDNA into cccDNA. In certain embodiments, the inhibitor detectably inhibits the formation and/or stability of the cccDNA as measured, e.g., using an assay described herein.

### RNA Destabilizer

In some embodiments, the compound described herein is used in combination with an RNA destabilizer. In some embodiments, an RNA destabilizer refers to a molecule, or a salt or solvate thereof, that reduces the total amount of HBV RNA in mammalian cell culture or in a live human subject. In a non-limiting example, an RNA destabilizer reduces the amount of the RNA transcript(s) encoding one or more of the following HBV proteins: surface antigen, core protein, RNA polymerase, and e antigen. In some embodiments, the RNA destabilizer is RG7834 or AB-452.

### Checkpoint Inhibitors

In some embodiments, the compound described herein is used in combination with a checkpoint inhibitor. In some embodiments, checkpoint inhibitors include any compound that is capable of inhibiting immune checkpoint molecules that are regulators of the immune system (e.g., stimulate or inhibit immune system activity). For example, some checkpoint inhibitors block inhibitory checkpoint molecules, thereby stimulating immune system function, such as stimulation of T cell activity against cancer cells. A non-limiting example of a checkpoint inhibitor is a PD-L1 inhibitor or a PD-1 inhibitor. In some embodiments, the PD-1 inhibitor is pembrolizumab, nivolumab, cemiplimab, spartalizumab, camrelizumab, sintilimab, tislelizumab, toripalimab, AMP-224, or AMP-514. In some embodiments, the PD-L1 inhibitor is atezolizumab, avelumab, durvalumab, KN035, CK-301, AUNP12, CA-170, or BMS-986189.

### Therapeutic Vaccines

In some embodiments, the compound described herein is used in combination with a therapeutic vaccine. In some embodiments, the therapeutic vaccine is HBsAG-HBIG, HB-Vac, ABX203, NASVAC, GS-4774, GX-110 (also known as HB-110E), CVI-HBV-002, RG7944 (also known as INO-1800), TG-1050, FP-02 (Hepsyn-B), AIC649, VGX-6200, KW-2, TomegaVax-HBV, ISA-204, NU-500, INX-102-00557 HBV MVA, or PepTcell.

### RNA Interference (RNAi) Therapeutics

In some embodiments, the compound described herein is used in combination with an RNA interference (RNAi) therapeutic. In some embodiments, the RNA interference therapeutic is TKM-HBV (also known as ARB-1467), ARB-1740, ARC-520, ARC-521, BB-HB-331, REP-2139, ALN-HBV, ALN-PDL, LUNAR-HBV, GS3228836, or GS3389404.

### HBV Entry Inhibitors

In some embodiments, the compound described herein is used in combination with an HBV entry inhibitor. In some embodiments, the HBV entry inhibitor is bulevirtide, IVIG-Tonrol, or GC-1 102.

### TLR Agonists

In some embodiments, the compound described herein is used in combination with a TLR agonist (TLR7, 8 and/or 9). In some embodiments, the TLR agonist is RG7795, GS-9620, SM360320, or AZD 8848.

### RIG-I Agonists

In some embodiments, the compound described herein is used in combination with a RIG-I agonist. In some embodiments, the RIG-I agonist is inarigivir.

### Interferons

In some embodiments, the compound described herein is used in combination with an interferon. In some embodiments, the interferon is interferon alpha (IFN-a), interferon alpha-2a, recombinant interferon alpha-2a, peginterferon alpha-2a, interferon alpha-2b, recombinant interferon alpha-2b, interferon alpha-2b XL, peginterferon alpha-2b, glycosylated interferon alpha-2b, interferon alpha-2c, recombinant interferon alpha-2c, interferon beta, interferon beta-1a, peginterferon beta-1a, interferon delta, interferon lambda (IFN-1), peginterferon lambda-1, interferon omega, interferon tau, interferon gamma (IFN-g), interferon alfacon-1, interferon alpha-n1, interferon alpha-n3, albinterferon alpha-2b, BLX-883, DA-3021, PI 101 (also known as AOP2014), PEG-infergen, belerofon, INTEFEN-IFN, albumin/interferon alpha 2a fusion protein, rHSA-IFN alpha 2a, rHSA-IFN alpha 2b, PEG-IFN-SA, interferon alpha biobetter; in particular, peginterferon alpha-2a, peginterferon alpha-2b, glycosylated interferon alpha-2b, peginterferon beta-1a, or peginterferon lambda-1.

### Preparation of Compounds

### Example 1: Synthesis of N-(3,4-difluorophenyl)-1,2,6,6-tetramethyl-8-oxo-1,4,5,6,7,8-hexahydropyrrolo[2,3-c]azepine-3-carboxamide

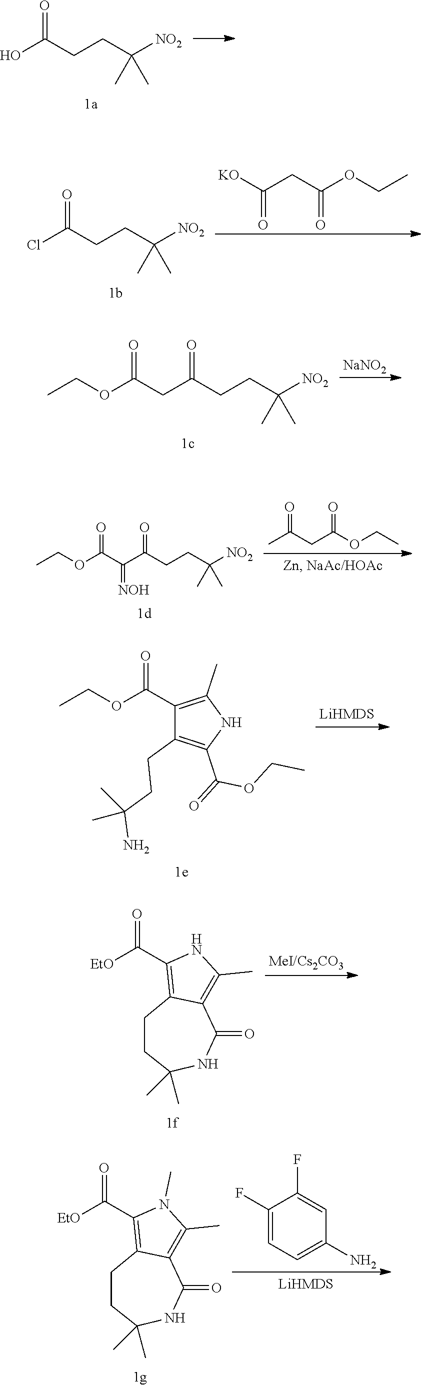

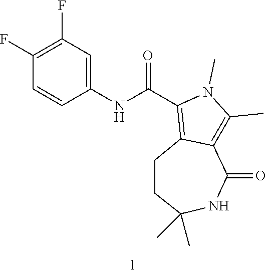 [SMILES: Cc1c2c(c(C(=O)Nc3ccc(F)c(F)c3)n1C)CCC(C)(C)NC2=O]

### Step 1: Synthesis of 4-methyl-4-nitropentanoyl chloride (1b)

Oxalyl chloride (2.4 mL) was added dropwise to a solution of 1a (1.5 g, 0.9 mmol) in DCM (10 mL) at RT, followed by the addition of DMF (30 μL). The reaction mixture was stirred at rt for 2 hrs, then, the solvent was removed in vacuo to afford crude 1b as yellow oil.

### Step 2: Synthesis of ethyl 6-methyl-6-nitro-3-oxoheptanoate (1c)

Triethylamine (3 mL, 24 mmol) was added dropwise to a suspension of potassium ethyl malonate (4.5 g, 26.4 mmol) in acetonitrile (60 mL) at 10° C., followed by the addition of anhydrous MgCl₂ (2.5 g, 26.5 mmol) over 30 min. The reaction mixture was warmed to rt for 2 hrs. The resulting white slurry was cooled to 0° C., and the crude 1a (1.5 g, in 6 mL of CH₃CN) was added dropwise over a period of 20 min. The mixture was warmed to rt overnight. The solvent was evaporated in vacuo and the residue was dissolved in toluene, cooled with ice-water (<5° C.), and acidified with 1 N HCl to pH ˜2. After stirring for 15 min., the aqueous phase was separated and the organic phase was washed with 1 N HCl, water and brine, concentrated and purified by column (EtOAc/hexanes 0 100%) to give 1c as colorless oil (1.4 g). ESI-MS, m/z 232.1 [M+H]⁺.

### Step 3: Synthesis of ethyl 2-(hydroxyimino)-6-methyl-6-nitro-3-oxoheptanoate (1d)

NaNO₂ (0.45 g in water 4 mL, 6.5 mmol) was added dropwise to a solution of 1c (1.4 g in AcOH 4 mL) at 3˜7° C. After 30 min, the reaction mixture was warmed to RT for 1 h. Water (10 mL) was added, and extracted with EtOAc (2×15 mL). The combined organic layer was washed with water, brine, and concentrated to afford crude id as colorless oil. ESI-MS, m/z 283.1 [M+23]⁺.

### Step 4: Synthesis of diethyl 3-(3-amino-3-methylbutyl)-5-methyl-1H-pyrrole-2,4-dicarboxylate (1e)

Zn dust (1.5 g, 23 mmol) was added portionwise to a solution of id (1.5 g, 5.7 mmol), ethyl acetonacetate (1.0 g, 7.7 mmol) and NaOAc (0.8 g) in AcOH (10 mL) at 70˜80° C. in a period of 30 min. After 2 h at 75° C., the reaction mixture was cooled to RT, and filtered. The filtrate was concentrated in vacuo, and the residue was purified by reverse phase chromatography eluted with ACN and water, and dried using lyophilization to afford the title product as white solid (0.45 g). ESI-MS, m/z 311.1 [M+H]⁺.

### Step 5: Synthesis of ethyl 2,6,6-trimethyl-8-oxo-1,4,5,6,7,8-hexahydropyrrolo[2,3-c]azepine-3-carboxylate (1f)

Lithium bis(trimethylsilyl)amide (1 N in THF, 10 mL) was added dropwise to a solution of 1e (TFA-salt, 0.5 g, 1.2 mmol) in THF (10 mL) at 0° C. The mixture was warmed to RT for 2 h, then, heated at 80° C. for 12 h. The reaction mixture was quenched with NH₄Cl (sat. aq.) at 0° C. The aqueous layer was extracted with EtOAc (2×15 mL). The combined organic layers were concentrated and the residue was purified by reverse phase chromatography eluted with ACN and water, and dried using lyophilization to afford the title product white solid (0.1 g). ¹H NMR (300 MHz, CDCl₃) δ 9.3 (br s, 1H), 6.0 (br s, 1H), 4.32 (q, 2H, J=6.9 Hz), 3.19 (t, 2H, J=6.0 Hz), 2.59 (s, 3H), 1.97 (t, 2H, J=6.6 Hz), 1.37 (t, 3H, J=7.0 Hz), 1.30 (s, 6H). ESI-MS, m/z 265.0 [M+H]⁺.

### Step 6: Synthesis of ethyl 2,3,6,6-tetramethyl-4-oxo-2,4,5,6,7,8-hexahydropyrrolo[3,4-c]azepine-1-carboxylate (1g)

MeI (0.1 g, 0.7 mmol) was added to a mixture of 1f (50 mg, 0.18 mmol) and Cs₂CO₃ (0.2 g, 0.6 mmol) in DMF (0.5 mL) at 0° C. The mixture was warmed to 30° C. for 20 h. The mixture was quenched with water (4 mL), and extracted with EtOAc (2×5 mL). The combined organic layers were concentrated and the residue was purified by reverse phase chromatography eluted with ACN and water, and dried using lyophilization to afford the title product as white solid. ESI-MS, m/z 279.0 [M+H]⁺.

### Step 7: Synthesis of N-(3,4-difluorophenyl)-1,2,6,6-tetramethyl-8-oxo-1,4,5,6,7,8-hexahydropyrrolo[2,3-c]azepine-3-carboxamide

Lithium bis(trimethylsilyl)amide solution (1 M in THF, 1 mL, 10 mmol) was added dropwise over 5 min to a solution containing 1g (20 mg, 0.07 mmol) and 3,4-difluoroaniline (20 mg, 0.16 mmol) in THF (10 mL) at 0° C. The reaction mixture was allowed to warm slowly to RT. After 16 h, the reaction mixture was quenched with NH₄Cl (sat. aq.) and water, and extracted with EtOAc (3×). The combined organics were washed with brine, concentrated. The crude residue was suspended in 1:1 EtOAc/hexanes, stirred for 1 h at room temperature, and purified by reverse phase chromatography eluted with ACN and water, and dried using lyophilization to afford the title product as white solid. ESI-MS, m/z 362.1 [M+H]⁺.

### Example 2. N-(3,4-difluorophenyl)-1,2,6,6-tetramethyl-8-oxo-1,4,5,6,7,8-hexahydropyrrolo[2,3-c]azepine-3-carboxamide

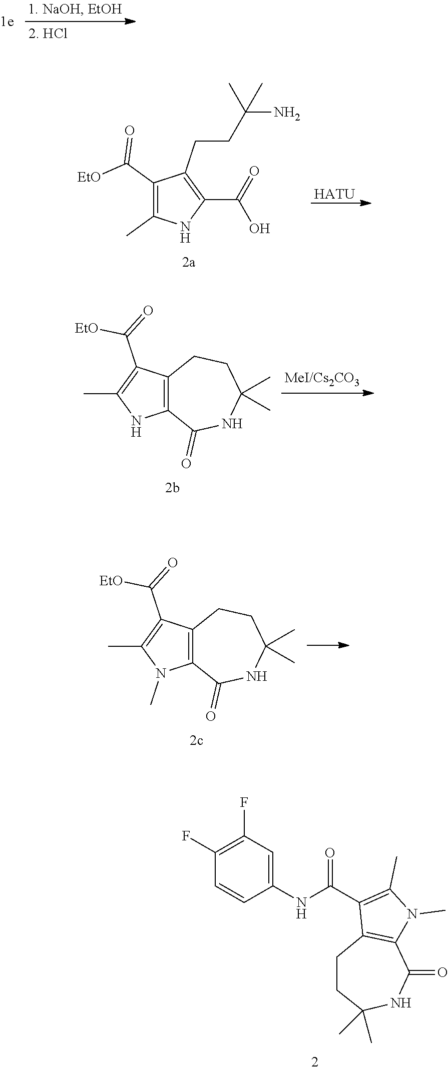

### Step 1: Synthesis of 3-(3-amino-3-methylbutyl)-4-(ethoxycarbonyl)-5-methyl-1H-pyrrole-2-carboxylic acid (2a)

NaOH (2N aq., 1 mL) was added to a solution of 1e (0.1 g, 0.32 mmol) at 0° C. in EtOH. The mixture was warmed to RT for 4 h, then at 45° C. for 72 h. The reaction mixture was cooled to RT, diluted with EtOAc and neutralized to pH ˜2 with HCl (1 N aqueous). The solvent was removed in vacuo and the residue was dried using lyophilization to afford the title product as off-white solid: ESI-MS, m/z 283.1 [M+H]⁺.

### Step 2: Synthesis of ethyl 2,6,6-trimethyl-8-oxo-1,4,5,6,7,8-hexahydropyrrolo[2,3-c]azepine-3-carboxylate (2b)

DIPEA was added to a solution of 2a (30 mg, 0.1 mmol) and HATU (50 mg, 0.13 mmol) in DMA at RT. After 4 h, the reaction was quenched with 4% TFA in water, and purified by reverse-phase chromatography (eluted with ACN and water), and dried using lyophilization to afford the title product as white solid (12 mg). ¹H NMR (300 MHz, CDCl₃) δ 9.7 (br s, 1H), 6.0 (br s, 1H), 4.28 (2H, J=6.9 Hz), 3.12 (2H, J=6.0 Hz), 2.51 (s, 3H), 1.94 (t, 2H, J=6.6 Hz), 1.35 (t, 3H, J=6.9 Hz), 1.29 (s, 6H). ESI-MS, m/z 265.1 [M+H]⁺.

### Step 3: Synthesis of ethyl 1,2,6,6-tetramethyl-8-oxo-1,4,5,6,7,8-hexahydropyrrolo[2,3-c]azepine-3-carboxylate (2c)

The title compounds was prepared following the procedure described in Example 1, Step 6, using 2b instead of 1f. ESI-MS, m/z 279.1 (M+H)⁺.

### Step 4: Synthesis of N-(3,4-difluorophenyl)-1,2,6,6-tetramethyl-8-oxo-1,4,5,6,7,8-hexahydropyrrolo[2,3-c]azepine-3-carboxamide

The title compound was prepared following the procedure described in Example 1, Step 7, using 2c instead of 1g. ESI-MS, m/z 362.1 [M+H]⁺.

### Example 3. 2-ethyl-N-(4-fluoro-3-methylphenyl)-3,6,6-trimethyl-4-oxo-2,4,5,6,7,8-hexahydropyrrolo[3,4-c]azepine-1-carboxamide

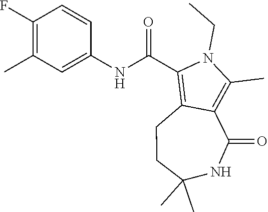 [SMILES: CCn1c(C)c2c(c1C(=O)Nc1ccc(F)c(C)c1)CCC(C)(C)NC2=O]

The title compound was prepared following the procedure described in Example 1, Step 6 and Step 7, using iodoethane and 4-fluoro-3-methylaniline. ESI-MS, m/z 372.2 [M+H]⁺.

### Example 4. N-(4-fluoro-3-methylphenyl)-2,3,6,6-tetramethyl-4-oxo-2,4,5,6,7,8-hexahydropyrrolo[3,4-c]azepine-1-carboxamide

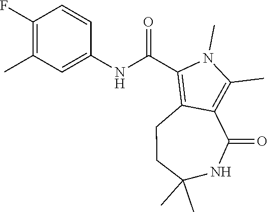 [SMILES: Cc1cc(NC(=O)c2c3c(c(C)n2C)C(=O)NC(C)(C)CC3)ccc1F]

The title compound was prepared following the procedure described in Example 1, Step 7, using 4-fluoro-3-methylaniline instead of 3,4-difluoroaniline. ESI-MS, m/z 358.1 [M+H]⁺.

### Example 5. 2,3,6,6-tetramethyl-4-oxo-N-(3,4,5-trifluorophenyl)-2,4,5,6,7,8-hexahydropyrrolo[3,4-c]azepine-1-carboxamide

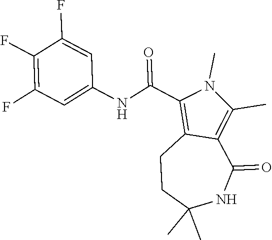 [SMILES: Cc1c2c(c(C(=O)Nc3cc(F)c(F)c(F)c3)n1C)CCC(C)(C)NC2=O]

The title compound was prepared following the procedure described in Example 1, Step 7, using 3,4,5-trifluoroaniline instead of 3,4-difluoroaniline. ESI-MS, m/z 380.2 [M+H]⁺.

### Example 6. N-(6-fluoro-5-methylpyridin-3-yl)-2,3,6,6-tetramethyl-4-oxo-2,4,5,6,7,8-hexahydropyrrolo[3,4-c]azepine-1-carboxamide

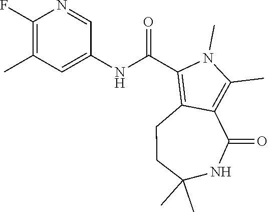 [SMILES: Cc1cc(NC(=O)c2c3c(c(C)n2C)C(=O)NC(C)(C)CC3)cnc1F]

The title compounds were prepared following the procedure described in Example 1, 7 using 6-fluoro-5-methylpyridin-3-amine instead of 3,4-difluoroaniline. ESI-MS, m/z 359.2 (M+H)⁺.

### Example 7. N-(2-fluoropyridin-4-yl)-2,3,6,6-tetramethyl-4-oxo-2,4,5,6,7,8-hexahydropyrrolo[3,4-c]azepine-1-carboxamide

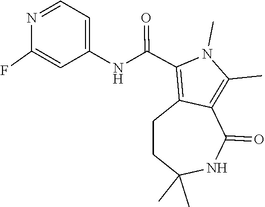 [SMILES: Cc1c2c(c(C(=O)Nc3ccnc(F)c3)n1C)CCC(C)(C)NC2=O]

The title compound was prepared following the procedure described in Example 1, Step 7, using 2-fluoropyridin-4-amine instead of 3,4-difluoroaniline, and the reaction mixture was heated at 80° C. for 5 h. After cooling to RT, the reaction mixture was quenched with NH₄Cl (sat. aq.), and extracted with EtOAc. The organic layer was separated, concentrated in vacuo and purified by reverse phase chromatography (eluted with ACN and water), and fractions combined and dried using lyophilization to afford the title product as white solid. ESI-MS, m/z 345.1 [M+H]⁺.

### Example 8. N-(3-chlorophenyl)-2,3,6,6-tetramethyl-4-oxo-2,4,5,6,7,8-hexahydropyrrolo[3,4-c]azepine-1-carboxamide

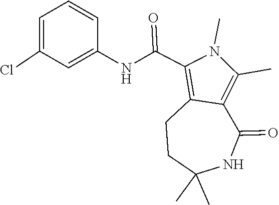 [SMILES: Cc1c2c(c(C(=O)Nc3cccc(Cl)c3)n1C)CCC(C)(C)NC2=O]

The title compound was prepared following the procedure described in Example 1, Step 7, using 3-chloroaniline instead of 3,4-difluoroaniline. ESI-MS, m/z 360.1 [M+H]⁺.

### Example 9. N-(3-(difluoromethyl)phenyl)-2,3,6,6-tetramethyl-4-oxo-2,4,5,6,7,8-hexahydropyrrolo[3,4-c]azepine-1-carboxamide

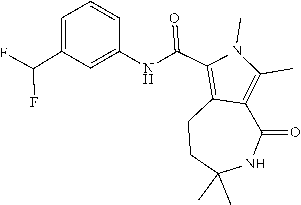 [SMILES: Cc1c2c(c(C(=O)Nc3cccc(C(F)F)c3)n1C)CCC(C)(C)NC2=O]

The title compound was prepared following the procedure described in Example 1, Step 7, using 3-(difluoromethyl)aniline instead of 3,4-difluoroaniline. ESI-MS, m/z 376.1 [M+H]⁺.

### Example 10. N-(2-fluoro-5-methylphenyl)-2,3,6,6-tetramethyl-4-oxo-2,4,5,6,7,8-hexahydropyrrolo[3,4-c]azepine-1-carboxamide

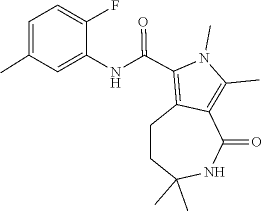

The title compound was prepared following the procedure described in Example 1, Step 7, using 2-fluoro-5-methylaniline instead of 3,4-difluoroaniline. ESI-MS, m/z 358.1 [M+H]⁺.

### Example 11. N-(3-fluoro-5-methylphenyl)-2,3,6,6-tetramethyl-4-oxo-2,4,5,6,7,8-hexahydropyrrolo[3,4-c]azepine-1-carboxamide

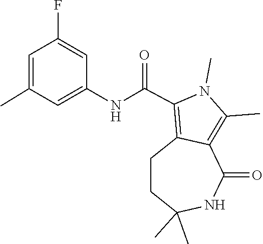

The title compound was prepared following the procedure described in Example 1, Step 7, using 3-fluoro-5-methylaniline instead of 3,4-difluoroaniline. ESI-MS, m/z 358.1 [M+H]⁺.

### Example 12. N-(3-(difluoromethyl)-4-fluorophenyl)-2,3,6,6-tetramethyl-4-oxo-2,4,5,6,7,8-hexahydropyrrolo[3,4-c]azepine-1-carboxamide

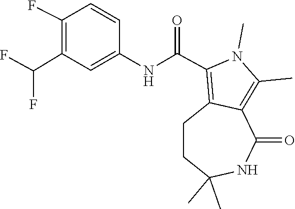 [SMILES: Cc1c2c(c(C(=O)Nc3ccc(F)c(C(F)F)c3)n1C)CCC(C)(C)NC2=O]

The title compound was prepared following the procedure described in Example 1, Step 7, using 3-(difluoromethyl)-4-fluoroaniline instead of 3,4-difluoroaniline. ESI-MS, m/z 394.1 [M+H]⁺.

### Example 22. N-(4-fluoro-3-methylphenyl)-2,3,6-trimethyl-7-methylene-4-oxo-4,5,6,7-tetrahydro-2H-pyrrolo[3,4-c]pyridine-1-carboxamide

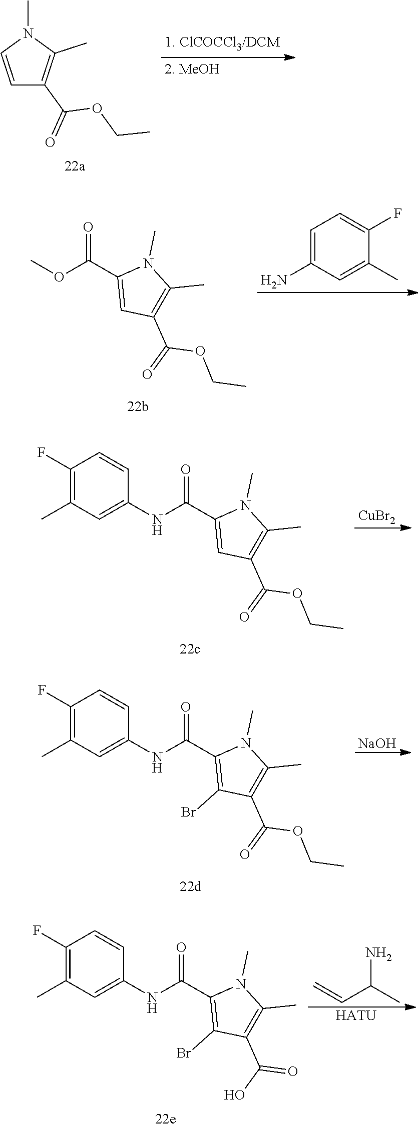

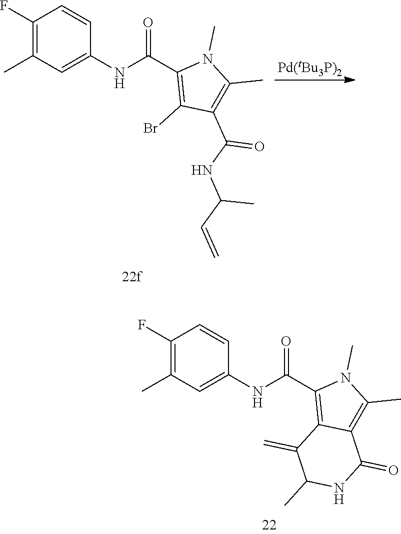

### Step 1: Synthesis of 4-ethyl 2-methyl 1,5-dimethyl-1H-pyrrole-2,4-dicarboxylate (22b)

Trichloroacetyl chloride (0.4 mL) was added to a solution of 22a (0.36 g, 2.2 mmol) in DCM (1 mL) at 0° C. The mixture was warmed to RT overnight. The reaction mixture was concentrated. The residue was dissolved in MeOH, then, NaOMe (˜25% by weight in MeOH, 0.4 mL) was added at 0° C. After 2 h, the reaction mixture was quenched with water, and extracted with EtOAc. The organic layer was concentrated and purified by flash chromatography (EtOAc/hexanes 0 100%) to give 22b as a white solid (0.24 g). ¹H NMR (300 MHz, CDCl₃) δ 7.37 (s, 1H), 4.29 (q, J=7.2 Hz, 2H), 3.88 (s, 3H), 3.84 (s, 3H), 2.58 (s, 3H), 1.36 (t, J=6.8 Hz, 3H). ESI-MS, m/z 226.1 [M+H]⁺.

### Step 2: Synthesis of ethyl 5-((4-fluoro-3-methylphenyl)carbamoyl)-1,2-dimethyl-1H-pyrrole-3-carboxylate (22c)

LiHMDS (1 N in THF, 20 mL) was added to a solution of 22b (2 g, 9 mmol) and 4-fluoro-3-methylaniline (1.4 g, 1.2 eq) in THF (20 mL) at 0° C. The mixture was warmed to rt overnight. The reaction mixture was quenched with sat. NH₄Cl aqueous, extracted with EtOAc, concentrated and purified by flash chromatography (EtOAc/hexanes 0˜100%) to give 22c as white solid (1.6 g): ESI-MS, m/z 319.2 [M+H]⁺.

### Step 3: Synthesis of ethyl 4-bromo-5-((4-fluoro-3-methylphenyl)carbamoyl)-1,2-dimethyl-1H-pyrrole-3-carboxylate (22d)

CuBr₂ (30 mg, 0.14 mmol) was added to a solution of 22c (30 mg, 0.1 mmol) in CH₃CN (1 mL) at 0° C. The mixture was warmed to rt overnight. The reaction was quenched with sat. NH₄Cl, and extracted with EtOAc. The organic layer was concentrated and purified by flash chromatography (EtOAc/hexanes 0˜100%) to give 22d as white solid (15 mg). ¹H NMR (300 MHz, CD₃OD) δ 7.4-7.5 (m, 2H), 7.01 (t, J=9.2 Hz, 1H), 4.29 (q, J=6.8 Hz, 2H), 3.68 (s, 3H), 2.55 (s, 3H), 2.27 (s, 3H), 1.37 (t, J=6.8 Hz, 3H). ESI-MS, m/z 397.0, 399.0 [M+H]⁺.

### Step 4: Synthesis of 4-bromo-5-((4-fluoro-3-methylphenyl)carbamoyl)-1,2-dimethyl-1H-pyrrole-3-carboxylic acid (22e)

NaOH (2 N aqueous, 0.3 mL, 0.06 mmol) and 22d (10 mg, 0.02 mmol) in EtOH (0.3 mL) was heated at 80° C. for 20 h. The mixture was cooled to RT, neutralized to pH ˜3 with 2 N aq. HCl, concentrated, and the residue was purified by reverse phase chromatography (eluted with ACN and water), and dried using lyophilization to afford 22e as white solid. ¹H NMR (300 MHz, CD₃OD) δ 9.95 (s, 1H), 7.52-7.64 (m, 2H), 7.09 (d, J=8.8 Hz, 1H), 3.54 (s, 3H), 2.22 (s, 3H). ESI-MS, m/z 369.0, 371.0 [M+H]⁺.

### Step 5: Synthesis of 3-bromo-N4-(but-3-en-2-yl)-N2-(4-fluoro-3-methylphenyl)-1,5-dimethyl-1H-pyrrole-2,4-dicarboxamide (22f)

DIPEA (10 mg) was added to a mixture of 22e (15 mg) and HATU (20 mg) in DMA (0.4 mL) at 0° C. The mixture was warmed to RT overnight. The mixture was quenched with 4% aq. TFA, then purified by reverse phase chromatography eluted with ACN and water, and dried using lyophilization to afford 22f white solid (12 mg). ESI-MS, m/z 423.1 [M+H]⁺.

### Step 6: Synthesis of N-(4-fluoro-3-methylphenyl)-2,3,6-trimethyl-7-methylene-4-oxo-4,5,6,7-tetrahydro-2H-pyrrolo[3,4-c]pyridine-1-carboxamide (22)

A mixture of 22f (10 mg), (^{t}Bu₃P)₂Pd (4 mg) and TEA (50 mg) in DMF (0.4 mL) was flushed with argon, then heated under microwave at 120° C. for 25 min. After cooling to rt, the reaction mixture was quenched with NH₄Cl (sat. aq.), and extracted with EtOAc. The organic layer was separated, concentrated and the residue was purified by reverse phase chromatography (eluted with ACN and water), and dried using lyophilization to afford the title product as white solid: ESI-MS, m/z 342.2 [M+H]⁺.

### Example 23. N-(4-fluoro-3-methylphenyl)-2,3,6,7-tetramethyl-4-oxo-4,5,6,7-tetrahydro-2H-pyrrolo[3,4-c]pyridine-1-carboxamide

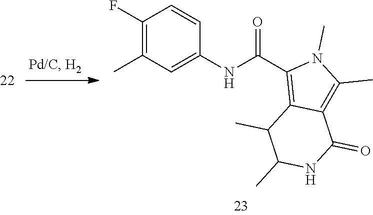

A mixture of Pd/C (10%, 20 mg) and 22 (10 mg) in MeOH (4 mL) was flushed with argon, then, stirred under hydrogen (balloon) at rt for 20 h. The reaction mixture was quenched with 4% aq. TFA, filtered, concentrated and the residue was purified by reverse phase chromatography (eluted with ACN and water), and dried using lyophilization to afford the title product as white solid. ESI-MS, m/z 344.2 [M+H]⁺.

### Example 24. N-(4-fluoro-3-methylphenyl)-2,3,6-trimethyl-4-oxo-2,4,5,6,7,8-hexahydropyrrolo[3,4-c]azepine-1-carboxamide

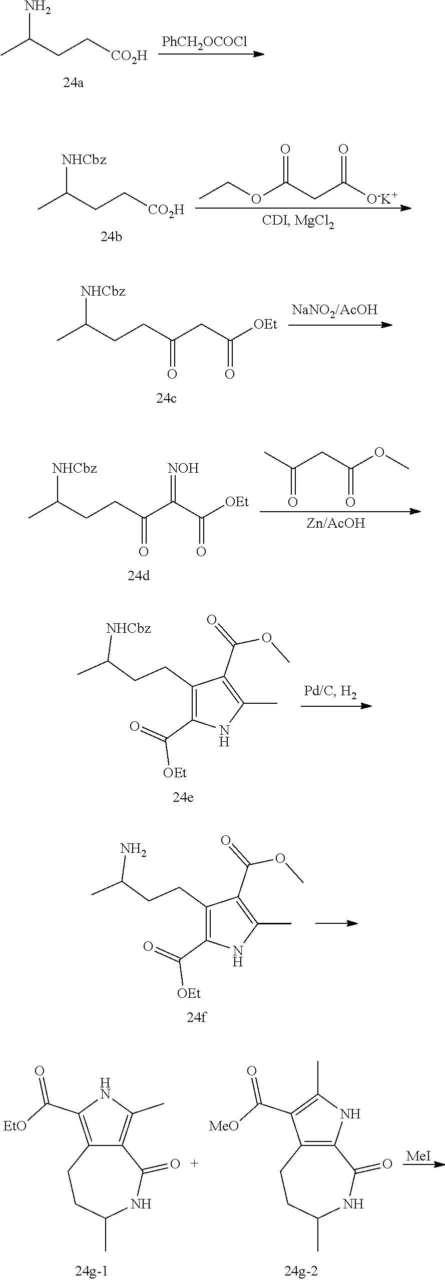

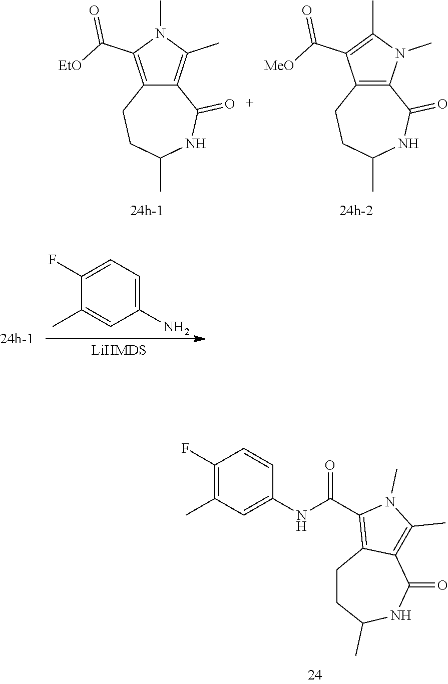

### Step 1: Synthesis of 4-(((benzyloxy)carbonyl)amino)pentanoic acid (24b)

KOH (2.0 g, 36 mmol) was added in portions to a mixture of 24a (2 g, 17 mmol) in water (10 mL) at 0˜8° C. After 30 min a solution of benzyl chloroformate (2.9 g, 17 mmol) in acetone (8 mL) was added. The mixture was allowed to warm to RT overnight. The reaction mixture was extracted with toluene (2×10 mL). The aqueous layer was cooled with ice-water, and neutralized to pH ˜2 with HCl (2 N aq.). The solid was collected, and dried to give 24b as white solid (1.4 g). ESI-MS, m/z 252.1 (M+H)⁺.

### Step 2: Synthesis of ethyl 6-(((benzyloxy)carbonyl)amino)-3-oxoheptanoate (24c)

CDI (0.8 g, 5.7 mmol) was added to a solution of 24a (1.4 g, 5.5 mmol) in THF (10 mL) at RT. After 1 h, mono-ethyl malonate potassium salt (1.4 g, 8.2 mmol) and MgCl₂ (1.2 eq.) was added to the solution. The mixture was stirred at RT overnight. The reaction mixture was diluted with EtOAc (20 mL), and neutralized to pH ˜2 (2 N HCl aq.) at 0˜8° C. The aqueous layer was extracted with EtOAc (2×20 mL). The combined organic layers was washed with water, brine, concentrated and purified by flash chromatography (EtOAc/hexanes 0˜100%) to give 24c as white solid (1.2 g). ESI-MS, m/z 321.1 [M+H]⁺.

### Step 3: Synthesis of ethyl (E)-6-(((benzyloxy)carbonyl)amino)-2-(hydroxyimino)-3-oxoheptanoate (24d)

NaNO₂ (0.3 g, dissolved in 3 mL water) was added dropwise to a solution of 24c (1.2 g) in 3 mL of AcOH at 3˜7° C. After 2 h the reaction was quenched with water, and extracted with EtOAc (3×10 mL). The combined organic layer was washed with water, brine and concentrated to give crude 24d as colorless oil. ESI-MS, m/z 373.1 [M+23]⁺.

### Step 4: Synthesis of 2-ethyl 4-methyl 3-(3-(((benzyloxy)carbonyl)amino)butyl)-5-methyl-1H-pyrrole-2,4-dicarboxylate (24e)

Zn dust (0.7 g, 10.7 mmol) was added to a solution of crude 24d (˜3.7 mmol), methyl acetonacetate (1.0 g, 7.5 mmol) and NaOAc (0.8 g) in AcOH (10 mL) at 70˜80° C. in 10 min. After 1 h, the reaction was cooled to RT and quenched with sat. aq. NH₄Cl, and the resulting mixture was extracted with EtOAc (3×10 mL). The combined organic layer was washed with water, brine, concentrated and purified by silica column (EtOAc/hexanes 0˜100%) to give 24e as white solid (1.1 g). ¹H NMR (400 MHz, CDCl₃) δ 9.35 (s, 1H), 7.36 (br m, 5H), 5.1-5.2 (br, 3H), 4.31 (q, J=6.8 Hz, 2H), 3.8 (s, 3H), 3.78-3.9 (m, 1H), 3.08 (br m, 2H), 2.49 (s, 3H), 1.6-1.7 (m, 2H), 1.34 (t, J=7.2 Hz, 3H), 1.21 (d, J=5.6 Hz, 3H). ESI-MS, m/z 417.2 [M+H]⁺.

### Step 5: Synthesis of 2-ethyl 4-methyl 3-(3-aminobutyl)-5-methyl-1H-pyrrole-2,4-dicarboxylate (24f)

A mixture of 24e (1.1 g, 2.6 mmol) and Pd(OH)₂/C (20%, 0.25 g) in MeOH (80 mL) was flushed with argon, then stirred under hydrogen gas for 4 h. The reaction mixture was flushed with argon, then, quenched with HCl (˜1 N in Et₂O, 5 mL), then the catalyst was filtered, and the filtrate concentrated in vacuo. The residue was suspended in CH₃CN (4 mL), filtered and dried to give 24f as white solid (0.7 g). ¹H NMR (400 MHz, CD₃OD) δ4.32 (q, J=6.8 Hz, 2H), 3.82 (s, 3H), 3.1-3.25 (m, 3H), 2.46 (s, 3H), 1.84-1.87 (m, 2H), 1.32-1.37 (m, 6H). ESI-MS, m/z 283.1 [M+H]⁺.

### Step 6: Synthesis of ethyl 3,6-dimethyl-4-oxo-2,4,5,6,7,8-hexahydropyrrolo[3,4-c]azepine-1-carboxylate (13g-1) and methyl 2,6-dimethyl-8-oxo-1,4,5,6,7,8-hexahydropyrrolo[2,3-c]azepine-3-carboxylate (13g-2)

LiHMDS (2.5 mL, 1 N in THF) was added to a mixture of 13h (0.1 g, HCl-salt) in THF (3 mL) at 0° C. The mixture was warmed to RT for 1 h, and 83° C. for 2 h. The mixture was cooled to RT, quenched with NH₄Cl (sat. aq), and extracted with EtOAc. The combined organic layer was concentrated to give 13g-1 and 13g-2. ESI-MS, m/z 251.1 and 237.1 [M+H]⁺.

### Step 7: Synthesis of ethyl 2,3,6-trimethyl-4-oxo-2,4,5,6,7,8-hexahydropyrrolo[3,4-c]azepine-1-carboxylate (13h-1) and methyl 1,2,6-trimethyl-8-oxo-1,4,5,6,7,8-hexahydropyrrolo[2,3-c]azepine-3-carboxylate (13h-2)

MeI (0.25 mL) was added to a mixture of 13g-1 and 13g-2 (100 mg) and Cs₂CO₃ (0.3 g) in DMF (0.5 mL) and water (20 μL) at RT. The mixture was stirred at RT for 20 h. The mixture was cooled in an ice-water bath, then NaH (50 mg, 60% in mineral oil) was added. After 5 min, MeI (100 mg) was added. The mixture was warmed to 38° C. for 10 min. The reaction mixture was quenched with water at 0° C., and extracted with EtOAc. The organic layer was concentrated and purified by reverse phase HPLC to give 13h-1 (60 mg) and 13h-2 (18 mg) as white solids. 13h-1: ¹H NMR (400 MHz, CD₃OD, 200-8) δ 4.29 (q, J=7.2 Hz, 2H), 3.79 (s, 3H), 3.35-3.52 (m, 2H), 2.5-2.6 (m, 1H), 2.39 (s, 3H), 1.78-1.93 (m, 2H), 1.35 (t, J=6.8 Hz, 3H), 1.21 (d, J=6.8 Hz, 3H). 13h-2: ¹H NMR (400 MHz, CD₃OD, 200-8-BP) δ 3.78 (s, 3H), 3.71 (s, 3H), 3.3-3.5 (m, 2H), 2.6-2.7 (m, 1H), 2.51 (s, 3H), 1.9-2.0 (m, 1H), 1.7-1.8 (m, 1H), 1.24 (d, J=6.8 Hz, 3H).

### Step 8: Synthesis of N-(4-fluoro-3-methylphenyl)-2,3,6-trimethyl-4-oxo-2,4,5,6,7,8-hexahydropyrrolo[3,4-c]azepine-1-carboxamide (24)

The title compounds were prepared following the procedure described in Example 1, Step 7, using 4-fluoro-3-methylaniline and 24h-1. ESI-MS, m/z 344.2 (M+H)⁺.

### Example 25. N-(4-fluoro-3-methylphenyl)-1,2,6-trimethyl-8-oxo-1,4,5,6,7,8-hexahydropyrrolo[2,3-c]azepine-3-carboxamide

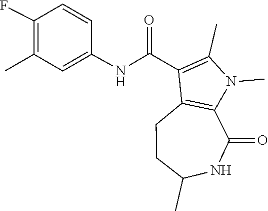 [SMILES: Cc1cc(NC(=O)c2c3c(n(C)c2C)C(=O)NC(C)CC3)ccc1F]

The title compound was prepared following the procedure described in Example 1, Step 7, using 4-fluoro-3-methylaniline and 24h-2. ESI-MS, m/z 344.2 (M+H)⁺.

### Example 45. N-(4-fluoro-3-methylphenyl)-2,3,6,7-tetramethyl-4-oxo-4,5-dihydro-2H-pyrrolo[3,4-c]pyridine-1-carboxamide

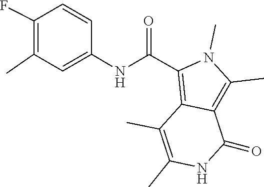 [SMILES: Cc1cc(NC(=O)c2c3c(C)c(C)[nH]c(=O)c3c(C)n2C)ccc1F]

The title compound was separated as by product from the reaction of example 23 as white solid: ESI-MS, m/z 342.2 [M+H]⁺.

### Example 46. (S)—N-(4-fluoro-3-methylphenyl)-2,3,6-trimethyl-4-oxo-4,5,6,7-tetrahydro-2H-pyrrolo[3,4-c]pyridine-1-carboxamide

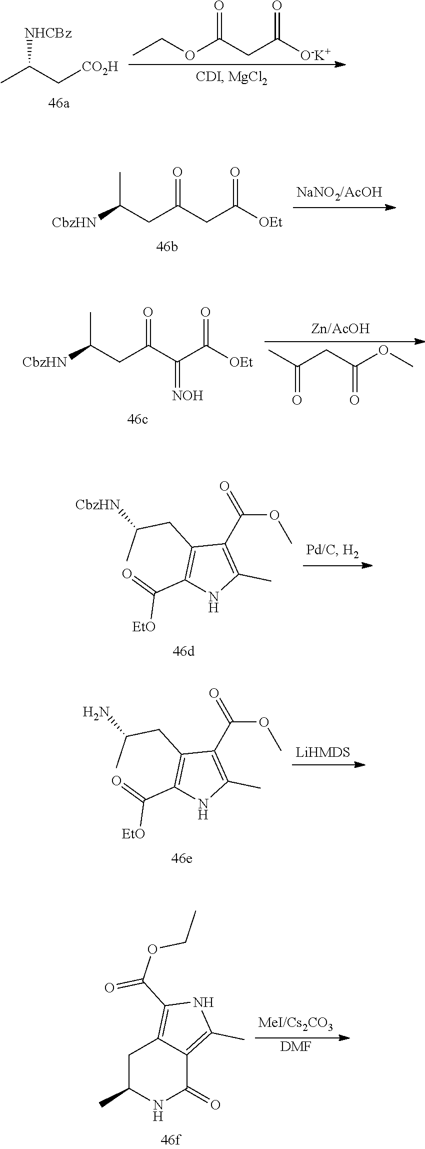

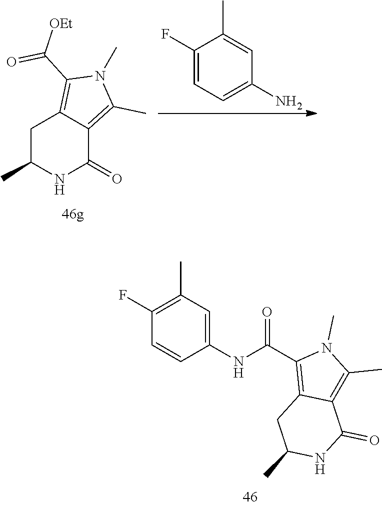

### Step 1: Synthesis of ethyl (S)-5-(((benzyloxy)carbonyl)amino)-3-oxohexanoate (46a)

CDI (0.7 g, 4.3 mmol) was added to a solution of 46a (1.0 g, 4.2 mmol) in THF (10 mL) at rt. After 1 h at RT, mono-ethyl malonate potassium salt (1.4 g, 8.2 mmol) and MgCl₂ (0.5 g, 5.3 mmol) was added to the solution. The mixture was stirred at RT overnight. The reaction was diluted with EtOAc (100 mL), and neutralized to pH ˜2 (2 N HCl aq.). The organic layers was washed with water, brine, concentrated and purified by silica column (EtOAc/hexanes 0˜100%) to give 46b as white solid (1.3 g). ¹H NMR (400 MHz, CDCl₃) δ 7.34 (br, 5H), 5.12 (s, 2H), 4.1-4.3 (m, 3H), 3.43 (s, 2H), 2.6-2.8.

### Step 2: Synthesis of ethyl (S)-5-(((benzyloxy)carbonyl)amino)-2-(hydroxyimino)-3-oxohexanoate (46b)

The title compound was prepared following the procedure described in Example 1, Step 3, using 46b instead of 1c. ESI-MS, m/z 337.1 (M+H)⁺.

### Step 3: Synthesis of 2-ethyl 4-methyl (S)-3-(2-(((benzyloxy)carbonyl)amino)propyl)-5-methyl-1H-pyrrole-2,4-dicarboxylate (46d)

The title compounds was prepared following the procedure described in Example 1, Step 4, using 46c instead of id. ESI-MS, m/z 403.1 (M+H)⁺.

### Step 4: Synthesis of 2-ethyl 4-methyl (S)-3-(2-aminopropyl)-5-methyl-1H-pyrrole-2,4-dicarboxylate (46e)

A mixture of 46d (1.5 g) in MeOH (80 mL) was flushed with argon, then, added Pd(OH)₂/C (10%, 0.25 g) under argon. The mixture was stirred under hydrogen for 4 h. The reaction was quenched with 1 N HCl in ether (5 mL), filtered over Celite, concentrated and lyophilized to give 46e as white solid (1.0 g). ¹H NMR (400 MHz, DMSO-d₆) δ 12.1 (s, 1H), 8.01 (br, 3H), 4.28 (q, 2H, J=6.8 Hz), 3.75 (s, 3H), 3.2-3.4 (m, 3H), 2.43 (s, 3H), 1.32 (t, 3H, J=7.2 Hz), 1.1 (br m, 3H).

### Step 5: Synthesis of ethyl (S)-3,6-dimethyl-4-oxo-4,5,6,7-tetrahydro-2H-pyrrolo[3,4-c]pyridine-1-carboxylate (46f)

LiHMDS (1 N in THF, 10 mL) was added dropwise to a mixture of 46e (0.6 g, 2.0 mmol) in THF (10 mL) at 0° C. under argon. The mixture was warmed to RT for 3 h. The reaction was quenched with sat. NH₄Cl at 0° C., and extracted with EtOAc (2×30 mL). The combined organic layers was washed with brine, dried over Na₂SO₄, concentrated to give 46f (0.5 g) as white solid. ESI-MS, m/z 236.1 (M+H)⁺.

### Step 6: Synthesis of ethyl (S)-2,3,6-trimethyl-4-oxo-4,5,6,7-tetrahydro-2H-pyrrolo[3,4-c]pyridine-1-carboxylate (46g)

MeI (0.6 g, 4.2 mmol) and 20 μL of water was added to a mixture of crude 46f (0.3 g, 1.1 mmol) and Cs₂CO₃ (1.0 g) in DMF (3 mL) at 0° C. The mixture was warmed to RT for 10 h, then the mixture was quenched with water and extracted with EtOAc (2×15 mL). The organic layer was washed with brine, concentrated and purified by flash chromatography column (EtOAc/hexanes 0 100%) to give 46g as white solid (0.25 g). ESI-MS, m/z 251.1 [M+H]⁺.

### Step 7: Synthesis of (S)—N-(4-fluoro-3-methylphenyl)-2,3,6-trimethyl-4-oxo-4,5,6,7-tetrahydro-2H-pyrrolo[3,4-c]pyridine-1-carboxamide

The title compound was prepared following the procedure described in Example 1, Step 7, using 46g and 4-fluoro-3-methylaniline. ESI-MS, m/z 330.1 (M+H)⁺.

### Example 47. (S)—N-(2-fluoro-3-methylphenyl)-2,3,6-trimethyl-4-oxo-4,5,6,7-tetrahydro-2H-pyrrolo[3,4-c]pyridine-1-carboxamide

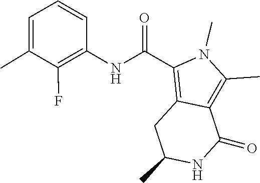

The title compound was prepared following the procedure described in Example 1, Step 7, using 46g and 2-fluoro-3-methylaniline. ESI-MS, m/z 330.1 (M+H)⁺.

### Example 48. N-(3,4-difluorophenyl)-2,3,6-trimethyl-4-oxo-2,4,5,6,7,8-hexahydropyrrolo[3,4-c]azepine-1-carboxamide

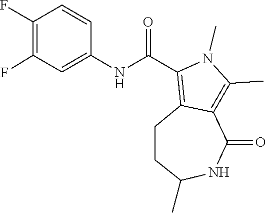 [SMILES: Cc1c2c(c(C(=O)Nc3ccc(F)c(F)c3)n1C)CCC(C)NC2=O]

The title compound was prepared following the procedure described in Example 24, using 24-1 and 3,4-difluoroaniline. ESI-MS, m/z 348.1 (M+H)⁺.

### Example I: Oral Composition of a Compounds of Formula (I), (Ta), or (Ib), or a Pharmaceutically Acceptable Salt, Solvate, or Stereoisomer Thereof

To prepare a pharmaceutical composition for oral delivery, 400 mg of compound described herein, or a pharmaceutically acceptable salt, solvate, or stereoisomer thereof and the following ingredients are mixed intimately and pressed into single scored tablets.

Tablet Formulation

| Ingredient | Quantity per tablet (mg) |
|---|---|
| compound | 400 |
| cornstarch | 50 |
| croscarmellose sodium | 25 |
| lactose | 120 |
| magnesium stearate | 5 |

The following ingredients are mixed intimately and loaded into a hard-shell gelatin capsule.

Capsule Formulation

| Ingredient | Quantity per capsule (mg) |
|---|---|
| compound | 200 |
| lactose spray dried | 148 |
| magnesium stearate | 2 |

### Example II: In Vitro Antiviral Assays

The anti-HBV activity of the Capsid Assembly Modulators (CAMs) was evaluated in a cell based assay utilizing the human hepatoma cell line HepAD38 (Ladner, S K., et al., 1998). HepAD38 cells were derived from the parental line, HepG2, that were stably transfected with a construct containing an HBV genome (genotype D, serotype ayw) under the control of a tetracycline repressible CMV promoter. Upon removal of tetracycline, viral pre-genomic RNA (pgRNA) and mRNAs are expressed and infectious viral particles are assembled and secreted into the culture medium providing a reliable, robust system to measure multiple steps of the HBV life cycle. Disruption of capsid formation results in reduced levels of DNA-containing virus particles that are released into the culture supernatant. To quantify the effect of CAMs on HBV replication, we developed a sensitive QPCR-based assay that measures extracellular HBV DNA levels upon treatment of HepAD38 cells with various concentrations of test compounds.

HepAD38 cells were maintained in DMEM/F12 medium containing 10% FBS, 400 μg/mL G418 and 0.3 μg/mL tetracycline (tet+media) to maintain repression of HBV replication. To evaluate each compound, HepAD38 cells were seeded into 24-well collagen coated culture plates (Corning BioCoat) at a density of 200,000 cells per well in 1 mL of medium without tetracycline (tet-media) and allowed to adhere overnight at 37° C., 5% CO₂ in a humidified incubator. The following day, media was refreshed and a dose range of each compound was prepared by performing 1 log₁₀ serial dilutions in 100% DMSO at 200×the desired assay concentration. Dilutions were then added to the cells resulting in a final dose range of 1 μM to 10 pM and the plates were returned to the incubator. Following 7 days of incubation, culture supernatants were harvested and HBV DNA levels were evaluated by QPCR and compared to the vehicle treated control wells (i.e. DMSO alone).

To quantify HBV DNA levels, cell culture supernatants were diluted 1:10 in sterile, nuclease-free water (Gibco). The diluted supernatants were subsequently added to a PCR master mix containing 1×Roche Light Cycler Master Mix, 0.5 μM forward primer, 0.5 μM reverse primer, 0.2 μM Roche Universal Probe Library Probe 25. The volume was brought to 20 μL with nuclease-free water and amplification of the HBV target sequence was performed using a Roche LightCycler 480 QPCR instrument. PCR extended out to 45 cycles with each cycle consisting of a denaturation step at 95° C. for 10 sec., followed by an annealing step at 60° C. for 10 sec. and a brief extension step at 72° C. for 1 sec.

Extracellular HBV DNA levels, expressed in copies/mL, were determined by comparison to a standard curve (10²-10⁹ copies/mL) using the Roche LightCycler analysis software. These values were subsequently converted to percent inhibition of HBV replication by dividing the HBV DNA levels in the experimental samples with those obtained from the vehicle control (˜1-2×105 copies/mL). Potency, expressed as an EC₅₀(the effective concentration required to inhibit 50% of HBV replication), was calculated from the dose-response curve using a 4-parameter non-linear regression analysis (GraphPad Prism). The nucleoside analog inhibitor entecavir was used as a positive control to validate each assay run. The EC₅₀ value of entecavir in the HepAD38 assay was 0.5 nM, as previously reported in the literature.

Table 2 summarizes the antiviral activity of the exemplary compounds. A: EC₅₀>1 μM; B: EC₅₀ values between 0.5 μM and 1 μM, inclusive; C: EC₅₀ values between 0.05 μM and 0.499 μM, inclusive; D: EC₅₀ values <0.05 μM. NT=not tested. NA=not applicable. PGP-1(

TABLE 2
Summary of anti-HBV replication in HepAD38 cells.

| | Anti-HBV | | Anti-HBV | | Anti-HBV | | Anti-HBV | | Anti-HBV |
|---|---|---|---|---|---|---|---|---|---|
| Ex. | EC₅₀ | Ex. | EC₅₀ | Ex. | EC₅₀ | Ex. | EC₅₀ | Ex. | EC₅₀ |
| 1 | D | 2 | C | 3 | D | 4 | D | 5 | A |
| 6 | A | 7 | C | 8 | C | 9 | C | 10 | D |
| 11 | D | 12 | D | 22 | B | 23 | D | 24 | D |
| 25 | D | 45 | B | 46 | D | 47 | B | 48 | B |

### Example III: In Vitro Cytotoxicity Assays

To evaluate antiviral selectivity, the cytotoxic activity of each compound was determined using a standard cell viability assay performed on the parental HepG2 cell line. Cell viability was determined by measuring the conversion of 3-(4,5-dimethylthiazol-2-yl)-2,5-diphenyltetrazolium bromide (MTT) to the insoluble formazan salt crystal that occurs in live cells. Briefly, HepG2 cells were seeded in 96-well plates at a density of 20,000 cells per well in EMEM+10% FBS (complete growth medium) and allowed to adhere overnight in a 37° C., 5% CO₂ humidified incubator. The next day, test agents were prepared by performing 8 half-log₁₀ serial dilutions in 100% DMSO at 200×the final desired concentration in the assay. Compounds were tested over a range of concentrations from 30 μM to 1.0 nM in the assay. HepG2 cells were incubated in the presence of various concentrations of CAMs for 7 days in a 37° C., 5% CO₂ humidified incubator. At the completion of the 7-day incubation period, MTT reagent was added to each well and the mixture was incubated for an additional 3-4 hours. At the completion of the incubation period, all wells were aspirated to remove the culture medium. The formazan crystals were solubilized from the cell monolayers with 100% DMSO. Plates were briefly mixed on an orbital shaker and absorbance was measured at 492 nm using a Perkin-Elmer EnVision multi-label plate reader. All absorbance values were converted to a percentage of the signal obtained from the vehicle treated controls. Absorbance values at 492 nm are directly proportional to the number of viable cells present in the sample. A CC₅₀ value (cytotoxic concentration that results in loss of 50% cell viability) was calculated from the dose-response curve by 4-parameter, non-linear regression analysis using the GraphPad Prism software. The positive control compound, staurosporine, reduced the viability of HepG2 cells in a dose-dependent manner (CC₅₀=100 nM).

Table 3 summarizes the cytotoxicity assay data in the hepatocyte cell line HepG2 for the example compounds. A: CC₅₀>30 μM; B: CC₅₀ values between 5 μM and 30 μM, inclusive; C: CC₅₀ values between 0.5 μM and 4.99 μM, inclusive; D: CC₅₀ values <0.5 μM. NT=not tested.

TABLE 3
Summary of cytotoxicity results in
HepG2 cells for example compounds.

| | HepG2 | | HepG2 | | HepG2 | | HepG2 | | HepG2 |
|---|---|---|---|---|---|---|---|---|---|
| Ex. | CC₅₀ | Ex. | CC₅₀ | Ex. | CC₅₀ | Ex. | CC₅₀ | Ex. | CC₅₀ |
| 1 | A | 2 | B | 3 | A | 4 | A | 5 | A |
| 6 | A | 7 | A | 8 | A | 9 | A | 10 | A |
| 11 | A | 12 | A | 22 | C | 23 | A | 24 | A |
| 25 | A | 45 | B | 46 | A | 47 | A | 48 | A |

What is claimed is:

1. A compound of Formula (I), or a pharmaceutically acceptable salt, solvate, or stereoisomer thereof: 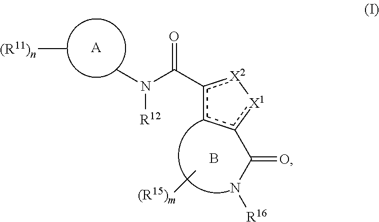
wherein:
Ring A is aryl, heteroaryl, cycloalkyl, or heterocycloalkyl;
each R¹¹ is independently halogen, —CN, —OH, —OR^{a}, —SH, —SR^{a}, —S(═O)R^{a}, —S(═O)₂R^{a}, —NO₂, —NR^{c}R^{d}, —NRS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is independently optionally substituted with one, two, or three R¹;
or two R¹¹ on adjacent atoms are taken together with the atoms to which they are attached to form a cycloalkyl, heterocycloalkyl, aryl, or heteroaryl; each optionally substituted with one, two, or three R²;
n is 0-4;
R¹² is hydrogen or C₁-C₆alkyl; 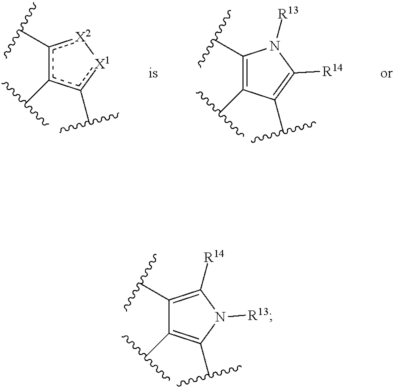
R¹³ is hydrogen, —S(═O)R^{a}, —S(═O)₂R^{a}, —S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein the alkyl, alkenyl, alkynyl, cycloalkyl, heterocycloalkyl, aryl, and heteroaryl is optionally substituted with one, two, or three R³;
R¹⁴ is hydrogen, halogen, —CN, —OH, —OR^{a}, —SH, —SW, —S(═O)R^{a}, —S(═O)₂R^{a}, —NO₂, —NR^{c}R^{d}, —NR^{b}S(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein the alkyl, alkenyl, alkynyl, cycloalkyl, heterocycloalkyl, aryl, and heteroaryl is optionally substituted with one, two, or three R⁴;
Ring B is a 6- to 9-membered heterocycloalkyl;
each R¹⁵ is independently halogen, —CN, —OH, —OR^{a}, —SH, —SR^{a}, —S(═O)R^{a}, —S(═O)₂R^{a}, —NO₂, —NR^{c}R^{d}, —NR^{b}S(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is independently optionally substituted with one, two, or three R⁵; or
two R¹⁵ on the same carbon atom are taken together to form an oxo, a C₂-C₆alkene, a cycloalkyl, or a heterocycloalkyl; wherein the cycloalkyl and heterocycloalkyl is optionally substituted with one, two, or three R⁶; or
two R¹⁵ on adjacent atoms are taken together to form a cycloalkyl or a heterocycloalkyl; wherein the cycloalkyl and heterocycloalkyl is optionally substituted with one, two, or three R⁷; or
two R¹⁵ on adjacent atoms are taken together to form a double bond;
m is 0-6;
R¹⁶ is hydrogen or C₁-C₆alkyl;
each R¹, R², R³, R⁴, R⁵, R⁶, and R⁷ is independently halogen, —CN, —OH, —OR^{a}, —SH, —SR^{a}, —S(═O)R^{a}, —S(═O)₂R^{a}, —NO₂, —NR^{c}R^{d}, —NR^{b}S(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl;
or two R¹, two R², two R³, two R⁴, two R⁵, two R⁶, or two R⁷ on the same carbon are taken together to form an oxo;
each R^{a} is independently C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl;
each R^{b} is independently hydrogen, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl;
or R^{c} and R^{d} are taken together with the atom to which they are attached to form a heterocycloalkyl optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl.

2. The compound of claim 1, or a pharmaceutically acceptable salt, solvate, or stereoisomer thereof, wherein:

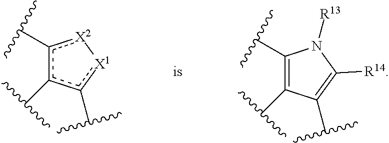

3. The compound of claim 1, or a pharmaceutically acceptable salt, solvate, or stereoisomer thereof, wherein:

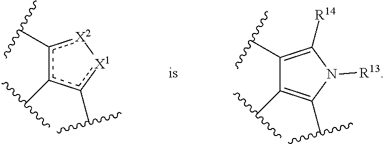

4. The compound of any one of claims 1-3, or a pharmaceutically acceptable salt, solvate, or stereoisomer thereof, wherein:
Ring A is aryl or heteroaryl.

5. The compound of any one of claims 1-4, or a pharmaceutically acceptable salt, solvate, or stereoisomer thereof, wherein:
Ring A is phenyl or pyridyl.

6. The compound of any one of claims 1-5, or a pharmaceutically acceptable salt, solvate, or stereoisomer thereof, wherein:
Ring A is phenyl.

7. The compound of any one of claims 1-6, or a pharmaceutically acceptable salt, solvate, or stereoisomer thereof, wherein:
each R¹¹ is independently halogen, —CN, —OH, —OR^{a}, —NR^{c}R^{d}, —C(═O)OR^{b}, —C(═O)NR^{c}R^{d}, C₁-C₆alkyl, C₁-C₆haloalkyl, or cycloalkyl.

8. The compound of any one of claims 1-7, or a pharmaceutically acceptable salt, solvate, or stereoisomer thereof, wherein:
each R¹¹ is independently halogen, —CN, C₁-C₆alkyl, or C₁-C₆haloalkyl.

9. The compound of any one of claims 1-8, or a pharmaceutically acceptable salt, solvate, or stereoisomer thereof, wherein:
each R¹¹ is independently halogen or C₁-C₆alkyl.

10. The compound of any one of claims 1-9, or a pharmaceutically acceptable salt, solvate, or stereoisomer thereof, wherein:
n is 0-3.

11. The compound of any one of claims 1-10, or a pharmaceutically acceptable salt, solvate, or stereoisomer thereof, wherein:
n is 1-3.

12. The compound of any one of claims 1-10, or a pharmaceutically acceptable salt, solvate, or stereoisomer thereof, wherein:
n is 2.

13. The compound of any one of claims 1-10, or a pharmaceutically acceptable salt, solvate, or stereoisomer thereof, wherein:
n is 3.

14. The compound of any one of claims 1-13, or a pharmaceutically acceptable salt, solvate, or stereoisomer thereof, wherein:
R¹² is hydrogen.

15. The compound of any one of claims 1-14, or a pharmaceutically acceptable salt, solvate, or stereoisomer thereof, wherein:
R¹³ is hydrogen, —S(═O)₂R^{a}, —S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, or cycloalkyl.

16. The compound of any one of claims 1-15, or a pharmaceutically acceptable salt, solvate, or stereoisomer thereof, wherein:
R¹³ is hydrogen, C₁-C₆alkyl, C₁-C₆haloalkyl, or C₁-C₆hydroxyalkyl.

17. The compound of any one of claims 1-16, or a pharmaceutically acceptable salt, solvate, or stereoisomer thereof, wherein:
R¹³ is C₁-C₆alkyl.

18. The compound of any one of claims 1-17, or a pharmaceutically acceptable salt, solvate, or stereoisomer thereof, wherein:
R¹⁴ is hydrogen, halogen, —CN, —OH, —OR^{a}, —NR^{c}R^{d}, —C(═O)OR^{b}, —C(═O)NR^{c}R^{d}, C₁-C₆alkyl, C₁-C₆haloalkyl, cycloalkyl, or —C₁-C₆alkyl(cycloalkyl).

19. The compound of any one of claims 1-18, or a pharmaceutically acceptable salt, solvate, or stereoisomer thereof, wherein:
R¹⁴ is hydrogen, halogen, or C₁-C₆alkyl.

20. The compound of any one of claims 1-19, or a pharmaceutically acceptable salt, solvate, or stereoisomer thereof, wherein:
R¹⁴ is hydrogen or C₁-C₆alkyl.

21. The compound of any one of claims 1-20, or a pharmaceutically acceptable salt, solvate, or stereoisomer thereof, wherein:
R¹⁴ is C₁-C₆alkyl.

22. The compound of any one of claims 1-21, or a pharmaceutically acceptable salt, solvate, or stereoisomer thereof, wherein:
Ring B is a 7- or 8-membered heterocycloalkyl.

23. The compound of any one of claims 1-22, or a pharmaceutically acceptable salt, solvate, or stereoisomer thereof, wherein:
Ring B is a 7-membered heterocycloalkyl.

24. The compound of any one of claims 1-22, or a pharmaceutically acceptable salt, solvate, or stereoisomer thereof, wherein:
Ring B is a 8-membered heterocycloalkyl.

25. The compound of any one of claims 1 or 22-24, or a pharmaceutically acceptable salt, solvate, or stereoisomer thereof, wherein:
the heterocycloalkyl of Ring B comprises 1, 2, or 3 heteroatoms selected from the group consisting of O, S, and N.

26. The compound of any one of claims 1 or 22-25, or a pharmaceutically acceptable salt, solvate, or stereoisomer thereof, wherein:
the heterocycloalkyl of Ring B comprises 1, 2, or 3 heteroatoms selected from the group consisting of O and N.

27. The compound of any one of claims 1 or 22-26, or a pharmaceutically acceptable salt, solvate, or stereoisomer thereof, wherein:
the heterocycloalkyl of Ring B comprises 1 or 2 heteroatoms selected from the group consisting of O and N.

28. The compound of any one of claims 1-27, or a pharmaceutically acceptable salt, solvate, or stereoisomer thereof, wherein: 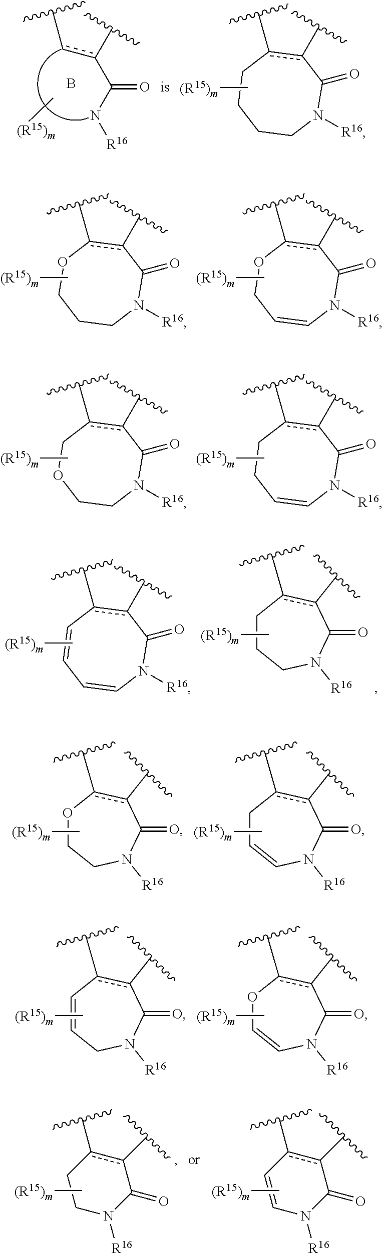
wherein [symbol] is a single bond or a double bond.

29. The compound of any one of claims 1-28, or a pharmaceutically acceptable salt, solvate, or stereoisomer thereof, wherein: 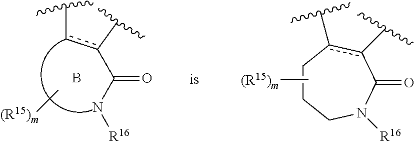
wherein [symbol] is a single bond or a double bond.

30. The compound of any one of claims 1-29, or a pharmaceutically acceptable salt, solvate, or stereoisomer thereof, wherein:
each R¹⁵ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, cycloalkyl, heterocycloalkyl, aryl, and heteroaryl is independently optionally substituted with one, two, or three R⁵.

31. The compound of any one of claims 1-30, or a pharmaceutically acceptable salt, solvate, or stereoisomer thereof, wherein:
each R¹⁵ is independently C₁-C₆alkyl, C₁-C₆haloalkyl, C₁-C₆hydroxyalkyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, cycloalkyl, heterocycloalkyl, aryl, and heteroaryl is independently optionally substituted with one, two, or three R⁵.

32. The compound of any one of claims 1-31, or a pharmaceutically acceptable salt, solvate, or stereoisomer thereof, wherein:
each R¹⁵ is independently C₁-C₆alkyl, C₁-C₆haloalkyl, or C₁-C₆hydroxyalkyl.

33. The compound of any one of claims 1-32, or a pharmaceutically acceptable salt, solvate, or stereoisomer thereof, wherein:
each R¹⁵ is independently C₁-C₆alkyl or C₁-C₆hydroxyalkyl.

34. The compound of any one of claims 1-33, or a pharmaceutically acceptable salt, solvate, or stereoisomer thereof, wherein:
each R¹⁵ is independently C₁-C₆alkyl.

35. The compound of any one of claims 1-29, or a pharmaceutically acceptable salt, solvate, or stereoisomer thereof, wherein:
two R¹⁵ on the same carbon atom are taken together to form an oxo, a cycloalkyl, or a heterocycloalkyl; wherein the cycloalkyl and heterocycloalkyl is optionally substituted with one, two, or three R⁵.

36. The compound of any one of claims 1-29 or 35, or a pharmaceutically acceptable salt, solvate, or stereoisomer thereof, wherein:
two R¹⁵ on the same carbon atom are taken together to form a cycloalkyl optionally substituted with one, two, or three R⁵.

37. The compound of any one of claims 1-29, or a pharmaceutically acceptable salt, solvate, or stereoisomer thereof, wherein:
two R¹⁵ on adjacent atoms are taken together to form a cycloalkyl or a heterocycloalkyl; wherein the cycloalkyl and heterocycloalkyl is optionally substituted with one, two, or three R⁷.

38. The compound of any one of claims 1-29 or 37, or a pharmaceutically acceptable salt, solvate, or stereoisomer thereof, wherein:
two R¹⁵ on adjacent atoms are taken together to form a heterocycloalkyl optionally substituted with one, two, or three R⁷.

39. The compound of any one of claims 1-38, or a pharmaceutically acceptable salt, solvate, or stereoisomer thereof, wherein:
m is 1-3.

40. The compound of any one of claims 1-39, or a pharmaceutically acceptable salt, solvate, or stereoisomer thereof, wherein:
m is 1 or 2.

41. The compound of any one of claims 1-40, or a pharmaceutically acceptable salt, solvate, or stereoisomer thereof, wherein:
R¹⁶ is hydrogen.

42. A compound of table 1, or a pharmaceutically acceptable salt, solvate, or stereoisomer thereof.

43. A pharmaceutical composition comprising a compound of any one of claims 1-42, or a pharmaceutically acceptable salt, solvate, or stereoisomer thereof, and a pharmaceutically acceptable excipient.

44. A method of treating an infection in a subject, comprising administering to the subject a compound of any one of claims 1-42, or a pharmaceutically acceptable salt, solvate, or stereoisomer thereof.

45. A method of treating an infection in a subject, comprising administering to the subject a pharmaceutical composition of claim 42.

46. The method of claim 44 or 45, wherein the infection is a viral infection.

47. The method of any one of claims 44-46, wherein the infection is caused by the hepatitis B virus.

48. The method of any one of claims 44-47, wherein the infection is hepatitis B.

49. The method of any one of claims 44-48, further comprising administering an additional therapeutic agent useful for treating a chronic HBV infection.

50. The method of claim 49, wherein the additional therapeutic agent useful for treating a chronic HBV infection is a reverse transcriptase inhibitor; an HBV polymerase inhibitor, a capsid inhibitor; a cccDNA formation inhibitor; an RNA destabilizer; a checkpoint inhibitor (e.g., PD-1/PD-L1 inhibitor); a therapeutic vaccine; an RNA interference (RNAi) therapeutic; an antisense-based therapeutic, an HBV entry inhibitor; a TLR agonist; an RIG-I agonist, or an interferon.
